(19) United States
(12) Patent Application Publication
(10) Pub. No.: US 2010/0197567 A1
(43) Pub. Date: Aug. 5, 2010

(54) CONUS POLYPEPTIDES

(75) Inventors: Andres Villu MARICQ, Salt Lake City, UT (US); Stori JENSEN, Salt Lake City, UT (US); Craig WALKER, Salt Lake City, UT (US); David MADSEN, West Valley City, UT (US); Baldomero OLIVERA, Salt Lake City, UT (US); Michael ELLISON, Thornton, CO (US)

Correspondence Address: ROTHWELL, FIGG, ERNST & MANBECK, P.C., 1425 K STREET, N.W., SUITE 800, WASHINGTON, DC, 20005, US

(73) Assignee: UNIVERSITY OF UTAH RESEARCH FOUNDATION, Salt Lake City, UT (US)

(21) Appl. No.: 12/580,487

(22) Filed: Oct. 16, 2009

Related U.S. Application Data
(60) Provisional application No. 61/106,212, filed on Oct. 17, 2008.

(51) Int. Cl. A61K 38/14; A61K 38/17; C07K 9/00; C07K 14/435; C07H 21/04; A61P 25/28; A61P 25/00; G01N 33/68

(52) U.S. Cl. 514/8; 514/12; 530/322; 530/324; 530/350; 536/23.5; 436/86

(57) ABSTRACT

The invention relates to peptides (termed propeller peptides herein) and related peptides (termed peptides of the G12.2 family herein), about 60 to about 90 residues in length, which are naturally available in minute amounts in the venom of the cone snails or analogous to the naturally available peptides, and which blocks the desensitization of AMPA-type ionotropic glutamate receptors (AMPARs).

### CROSS-REFERENCE TO RELATED APPLICATION

The present application is related to and claims priority under 35 U.S.C. §119(e) to U.S. provisional patent application Ser. No. 61/106,212 filed on 17 Oct. 2008, incorporated herein by reference.

### REFERENCE TO GOVERNMENT SUPPORT

This invention was made with Government support under Grant No. PO1 GM48677 and Grant No. MH067747 awarded by the National Institutes of Health, Bethesda, Md. The United States Government has certain rights in the invention.

### BACKGROUND OF THE INVENTION

The invention relates to peptides (termed propeller peptides herein) and related peptides (termed peptides of the G12.2 family herein), about 60 to about 90 residues in length, which are naturally available in minute amounts in the venom of the cone snails or analogous to the naturally available peptides, and which blocks the desensitization of AMPA-type ionotropic glutamate receptors (AMPARs).

The publications and other materials used herein to illuminate the background of the invention, and in particular, cases to provide additional details respecting the practice, are incorporated by reference, and for convenience are referenced in the following text by number and are listed by number in the appended bibliography.

The venom of marine gastropods in the genus Conus has yielded numerous structurally and functionally diverse peptidic components (1). The increasing variety of bioactive peptides identified in cone snail venoms has provided insight into the seemingly endless variety of directions taken by Conus species in evolving neuroactive molecules to suit their diverse biological purposes.

The bioactive peptides in Conus (“conopeptides”) are classified into two broad groups: the non-disulfide-rich and the disulfide-rich (1); the latter are conventionally called conotoxins. The non-disulfide-rich class includes conopeptides with no cysteines (contulakins (2), conantokins (3), and conorfamides (4)), and conopeptides with two cysteines forming a single disulfide bond (conopressins (5) and contryphans (6)). The conopeptides that comprise the disulfide-rich class have two or more disulfide bonds (1); among the major classes of molecular targets identified for these structurally diverse conopeptides are members of the voltage-gated and ligand-gated ion channel superfamilies.

However, the structure and function of only a small minority of these peptides have been determined to date. For peptides where function has been determined, three classes of targets have been elucidated: voltage-gated ion channels; ligand-gated ion channels, and G-protein-linked receptors.

Conus peptides which target voltage-gated ion channels include those that delay the inactivation of sodium channels, as well as blockers specific for sodium channels, calcium channels and potassium channels. Peptides that target ligand-gated ion channels include antagonists of NMDA and serotonin receptors, as well as competitive and noncompetitive nicotinic receptor antagonists. Peptides which act on G-protein receptors include neurotensin and vasopressin receptor agonists. The unprecedented pharmaceutical selectivity of conotoxins is at least in part defined by specific disulfide bond frameworks combined with hypervariable amino acids within disulfide loops (for a review see (24)).

Fast, excitatory neurotransmission in the vertebrate nervous system is primarily mediated by the neurotransmitter glutamate, which activates three distinct classes of pore-forming ionotropic receptors (iGluRs) that are distinguished on the basis of molecular and pharmacological criteria. Of these, the AMPA receptors (AMPARs) have a primary role in the transient depolarization of the postsynaptic membrane caused by synaptic release of glutamate (7). The time course of AMPAR-mediated changes of the synaptic potential is rapid, on the order of ms, and is influenced by both the rate of removal of glutamate from the synaptic cleft and by the processes of receptor inactivation and desensitization. AMPARs are tetrameric arrangements of 4 subunits arranged as a dimer of dimers. Binding of glutamate to the S1-S2 extracellular regions of an individual subunit causes a conformational change leading to pore opening (8, 9). In the continued presence of glutamate the receptor desensitizes with a conformational change of the receptor subunits (10). Recent studies indicate at least two classes of auxiliary proteins modulate the kinetics of AMPAR desensitization (11, 12, 13, 14).

In view of a large number of biologically active substances in Conus species it is desirable to further characterize them and to identify peptides capable of treating disorders involving voltage-gated ion channels, ligand-gated ion channels and/or receptors. Surprisingly, and in accordance with this invention, Applicants have discovered novel conopeptides that can be useful for the treatment of disorders involving AMPARs and could address a long felt need for a safe and effective treatment.

### SUMMARY OF THE INVENTION

The invention relates to peptides (termed propeller peptides herein) and related peptides (termed peptides of the G12.2 family herein), about 60 to about 90 residues in length, which are naturally available in minute amounts in the venom of the cone snails or analogous to the naturally available peptides, and which blocks the desensitization of AMPA-type ionotropic glutamate receptors (AMPARs).

More specifically, the present invention is directed to propeller peptides having the following formulas:

SGPADCCRMKECCTDRVNECLQRYSGREDKFVSFCYQEATVTCGSFNEIV
GCCYGYQMCMIRVVKPNSLSGAHEACKTVSCGNPCA
(SEQ ID NO: 1; C. striatus Con-ikot-ikot);
SPPPHNDCCKMKECCAQTTELCLKEFPNEEHIYTSTCYQRASHACGQFNE
IVGCCYGYRQCMLQNVQNLGLNWANQQCKEWNCLNPCE
(SEQ ID NO: 2; C. geographus Con-ikot-ikot);
SLPADCCNMKSCCTKSVYTCLQEYRGRENIFVTTCYQRASDICGSYNEIV
GCCYGYQMCMIQNVKPNQLLNAHVMCRNTDCNNPCQ
(SEQ ID NO: 3; C. bullatus Con-ikot-ikot);
QNRDCCIASIYQCLQRFPGQESYRAPPCQGEAVTECPNTDIDSCCPGYTT
CMSVHAQNNIRPAHNFCQNRLCYGP
(SEQ ID NO: 4; C. obscurus Con-ikot-ikot);
NPGCCPWEFYDCLIKRGVWKNRLYICYNMASRICAPGRSGGCCPMLLSCF
ERCRSRDSAVCYHRCKFASCWM
(SEQ ID NO: 5; C. pulicarius Con-ikot-ikot);
SLPADCCNMKSCCTKSVYTCLQVIVAVLLLTACQLITADDSRGTQLHRAL
RKATKLPVSTRCITPGTRCKVPSQCCRGPCKNGRCTPSPSEW
(SEQ ID NO: 16; C. bullatus 4_6_2 Con-ikot-ikot);
SRAGDCCRMKECCTDRVNTCLQGYSGREDVFVSFCYQEATHACGSFNEIV
GCCYGYQMCMIQAVKPGRLNPAHEACKTVACGKKKKKSLSSSPR
(SEQ ID NO: 17; C. circumcisus Con-ikot-ikot);
KRNDRSSRKCCAIKSYLCLQHHGCLSPINSNCAEQCKTTDTSGCGSTVGD
NCCSAYKSCLVDCQISRGDEHGDPLISCYNYCDQLHSC
(SEQ ID NO: 18; C. figulinus Con-ikot-ikot);
DLNIHKCCIRETHRCVRHCWDPHNPESDCVLDCYHREASHVCGTTGAGGC
CPGFVNCYGPCTMDADANLDVCRRRCKHEFCWDS
(SEQ ID NO: 19; C. planorbis Con-ikot-ikot);
DLSLDERNEPICCSWEMHGCLNQKRRWRLFLLICYREASMICPDGCCPDV
LSCFQECIPWDYDCYDSCSFVVC
(SEQ ID NO: 20; C. arenatus Con-ikot-ikot);
GLSLDERSVPGCCLWGVYDCLRGGGGWKSRLYYCYYDKASAYCTRGRPGG
CCPGLLRCFEDCTSPDSAVCFDRCKYVSC
(SEQ ID NO: 21; C. arenatus 1_1 Con-ikot-ikot);
NDRSSRKCCAVKSYLCLQGHGCLTPSDSSCAEQCKTTDTSTCGSNAGDNC
CSTYKSCLVDCQISRGNQHGDPLLICYNHCSQQRTYTGKWKVGIRWS
(SEQ ID NO: 22; C. betulinus Con-ikot-ikot);
and
NNGIRCCAIDSYQCLRDNGCLFPIDIDCAEQCKTTDTSYCGHAAGDCCFS
YKSCLVDCQIERGDEPGDQLRNCYNNCNSQGTYTGKWKVGIRWS
(SEQ ID NO: 23; C. virgo Con-ikot-ikot).

More specifically, the present invention is directed to peptides of the G12.2 family having the following formulas:

DESKCDRCNCAELRSSRCTQAIFCLTPELCTPSISCPTGECRCTKFHQSR
CTRFVECVPNKCRDA
(SEQ ID NO: 24; C. geographus 12.2);
DDSYCDGCLCTILKKETCTSTMSCRGTCRKEWPCWEEDCYCTEIQGGACV
TPSECKPGEC
(SEQ ID NO: 25; C. geographus 12.1);
DDSQCNECNCAHLKKAQCTDRIYCDFEAPCPSDYWCRNGKCLCARFHMGR
CSKSSECMPHQC
(SEQ ID NO: 26; C. erminius 12.1 3-3);
DENQCPECRCSELTTARCEESKYCHPDASCPTSSSCNNGKCLCSHFLGGR
CVSHSECNDSVC
(SEQ ID NO: 27; C. ventricosus 12.1);
DEAQCEECRCLELVIPECTERKYCHPEHSCPVRPYCNNGKCLCKHFFGGR
CAKRPDCNDSHCRAE
(SEQ ID NO: 28; C. arenatus 12);
DAGQCTGGTCRCSELKEAMCAESRLCLSPSCPSSSECADNHCLCTHILHG
VCVDSTECNPNKC
(SEQ ID NO: 29; C. erminius 12.2);
and
DESQCGDCRCFELRTARCTDKLQCESPDLCKPVVSCETGKCHCTRFSSGC
CTRTVECMPKKCF
(SEQ ID NO: 30; C. australis 26).

In addition, the present invention is directed to the above propeller peptides and peptides of the G12.2 family of the present invention in which the Pro residues may be substituted with hydroxyl-Pro; the Arg residues may be substituted by Lys, ornithine, homoargine, nor-Lys, N-methyl-Lys, N,N-dimethyl-Lys, N,N,N-trimethyl-Lys or any synthetic basic amino acid; the Lys residues may be substituted by Arg, ornithine, homoargine, nor-Lys, or any synthetic basic amino acid; the Tyr residues may be substituted with any synthetic hydroxy containing amino acid; the Ser residues may be substituted with Thr or any synthetic hydroxylated amino acid; the Thr residues may be substituted with Ser or any synthetic hydroxylated amino acid; the Phe and Trp residues may be substituted with any synthetic aromatic amino acid; and the Asn, Ser, Thr or Hyp residues may be glycosylated. The Tyr residues may also be substituted with the 3-hydroxyl or 2-hydroxyl isomers (meta-Tyr or ortho-Tyr, respectively) and corresponding O-sulpho- and O-phospho-derivatives or may be substituted with nor-Tyr, nitro-Tyr, mono-iodo-Tyr or di-iodo-Tyr. The aliphatic amino acids may be substituted by synthetic derivatives bearing non-natural aliphatic branched or linear side chains CₙH₂ₙ₊₂ up to and including n=8. The Leu residues may be substituted with Leu(D). The Trp residues may be substituted with halo-Trp, Trp(D) or halo-Trp(D). The halogen is iodo, chloro, fluoro or bromo; preferably iodo for halogen substituted-Tyr and bromo for halogen-substituted Trp. In addition, the halogen can be radiolabeled, e.g., ¹²⁵I-Tyr.

Examples of synthetic aromatic amino acid include, but are not limited to, such as nitro-Phe, 4-substituted-Phe wherein the substituent is C₁-C₃ alkyl, carboxyl, hyrdroxymethyl, sulphomethyl, halo, phenyl, —CHO, —CN, —SO₃H and —NHAc. Examples of synthetic hydroxy containing amino acid, include, but are not limited to, such as 4-hydroxymethyl-Phe, 4-hydroxyphenyl-Gly, 2,6-dimethyl-Tyr and 5-amino-Tyr. Examples of synthetic basic amino acids include, but are not limited to, N-1-(2-pyrazolinyl)-Arg, 2-(4-piperinyl)-Gly, 2-(4-piperinyl)-Ala, 2-[3-(2S)pyrrolininyl)-Gly and 2-[3-(2S)pyrrolininyl)-Ala. These and other synthetic basic amino acids, synthetic hydroxy containing amino acids or synthetic aromatic amino acids are described in Building Block Index, Version 3.0 (1999 Catalog, pages 4-47 for hydroxy containing amino acids and aromatic amino acids and pages 66-87 for basic amino acids; see also the website “amino-acids dot com”), incorporated herein by reference, by and available from RSP Amino Acid Analogues, Inc., Worcester, Mass.

Optionally, in the propeller peptides and peptides of the G12.2 family, the Asn residues may be modified to contain an N-glycan and the Ser, Thr and Hyp residues may be modified to contain an O-glycan (e.g., g-N, g-S, g-T and g-Hyp). In accordance with the present invention, a glycan shall mean any N-, S- or O-linked mono-, di-, tri-, poly- or oligosaccharide that can be attached to any hydroxy, amino or thiol group of natural or modified amino acids by synthetic or enzymatic methodologies known in the art. The monosaccharides making up the glycan can include D-allose, D-altrose, D-glucose, D-mannose, D-gulose, D-idose, D-galactose, D-talose, D-galactosamine, D-glucosamine, D-N-acetyl-glucosamine (GlcNAc), D-N-acetyl-galactosamine (GalNAc), D-fucose or D-arabinose. These saccharides may be structurally modified, e.g., with one or more O-sulfate, O-phosphate, O-acetyl or acidic groups, such as sialic acid, including combinations thereof. The gylcan may also include similar polyhydroxy groups, such as D-penicillamine 2,5 and halogenated derivatives thereof or polypropylene glycol derivatives. The glycosidic linkage is β and 1-4 or 1-3, preferably 1-3. The linkage between the glycan and the amino acid may be α or β, preferably α and is 1-.

Core O-glycans have been described by Van de Steen et al. (15), incorporated herein by reference. Mucin type O-linked oligosaccharides are attached to Ser or Thr (or other hydroxylated residues of the present peptides) by a GalNAc residue. The monosaccharide building blocks and the linkage attached to this first GalNAc residue define the “core glycans,” of which eight have been identified. The type of glycosidic linkage (orientation and connectivities) are defined for each core glycan. Suitable glycans and glycan analogs are described further in U.S. Pat. No. 6,369,193 and in PCT Published Application No. WO 00/23092, each incorporated herein by reference. A preferred glycan is Gal(β1→3)GalNAc(α1→).

The present invention is also directed to the identification of the nucleic acid sequences encoding these peptides and their propeptides and the identification of nucleic acid sequences of additional related propeller peptides and peptides of the G12.2 family. Thus, the present invention is directed to nucleic acids coding for the propeller peptide precursors (or propeller propeptides) and precursors of the peptides of the G12.2 family set forth herein. The present invention is further directed to the propeller propeptides set forth herein.

The present invention is further directed to a method of treating disorders associated AMPA-type ionotropic glutamate receptors (AMPARs) in a subject comprising administering to the subject an effective amount of the pharmaceutical composition comprising a therapeutically effective amount of a propeller peptide or a peptide of the G12.2 family described herein or a pharmaceutically acceptable salt or solvate thereof. The present invention is also directed to a pharmaceutical composition comprising a therapeutically effective amount of a propeller peptide described herein or a pharmaceutically acceptable salt or solvate thereof and a pharmaceutically acceptable carrier.

Another embodiment of the invention contemplates a method of identifying compounds that mimic the therapeutic activity of the instant peptides, comprising the steps of:

(a) conducting a biological assay on a test compound to determine the therapeutic activity; and
(b) comparing the results obtained from the biological assay of the test compound to the results obtained from the biological assay of the peptide. In relation to radioligand probes of propeller peptides and peptides of the G12.2 family for screening of small molecules, acting at unique allosteric sites, synthesis of such screening tools is not restricted to radioiodinated tyrosine derivatives. Incorporation of standard commercially available tritiated amino acid residues can also be utilized.

### BRIEF DESCRIPTION OF THE FIGURES

FIGS. 1a-1d show the isolation and identification of con-ikot-ikot. FIG. 1a: Glutamate-gated currents recorded from Xenopus oocytes that expressed GluR1(flop) before (−venom), and 1 minute (+venom) or 40 minutes after (40 min wash) the application of 0.18 mg venom from Conus striatus. Application of 1 mM glutamate indicated by bar. FIG. 1b: Final reverse phase HPLC separation of the active component (arrow). FIG. 1c: MALDI spectra of the active component. Peaks 1 and 2 correspond to the predicted molecular weights of the monomeric and dimeric peptides. FIG. 1d: Con-ikot-ikot amino acid sequence. Black box, predicted signal sequence; underline, propeptide; and gray box, sequence determined by Edman degradation.

FIGS. 2a-2e show active con-ikot-ikot toxin is a dimer of dimers. FIG. 2a: Reverse phase HPLC chromatogram of recombinant, column-purified con-ikot-ikot (see Methods). Three major fractions were isolated. FIG. 2b: Glutamate-gated currents recorded from Xenopus oocytes expressing GluR1(flop) and treated with fractions 1, 2 or 3 (6 μM). FIG. 2c: SDS-PAGE of native and recombinant toxin. Indicated are the fractions described above and the native toxin (N). Reducing conditions included 1 mM DTT. FIG. 2d: HPLC chromatograms showing fraction 1 and refolded fraction 1 (1R) of the recombinant toxin. Time of incubation (hrs) of fraction 1 in mixture of oxidized and reduced glutathione indicated next to trace. FIG. 2e: SDS-PAGE following RIPA buffer incubation of native and recombinant toxin. Reducing conditions included 1 mM DTT.

FIGS. 3a and 3b show the co-elution and activity of the recombinant protein. FIG. 3a: HPLC co-elution of native and recombinant con-ikot-ikot. FIG. 3b: Glutamate-gated current traces from Xenopus oocytes that expressed GluR1(flop) before and after application of 6 μM recombinant con-ikot-ikot.

FIGS. 4a and 4b show that Con-ikot-ikot specifically targets a subset of iGluRs. FIG. 4a: Current traces from Xenopus oocytes that express GluR1(flip), GluR1(flop), GluR3(flop), GluR4(flop), GluR6 and NR1/NR2A before (control) and after toxin application. FIG. 4b: Average relative increase of current in response to con-ikot-ikot application. *, p<0.01.

FIGS. 5a-5c show that Con-ikot-ikot potently enhances AMPAR-mediated current. FIG. 5a: Dose-response curve of glutamate-gated currents recorded from Xenopus oocytes that expressed GluR1(flip). FIG. 5b: Current-voltage curves before (control) and after con-ikot-ikot application. FIG. 5c: Glutamate-gated current in Xenopus oocytes that expressed an N-terminal deletion variant of GluR1(flip) (Δ60-471) both before (control) and after toxin application.

FIG. 6 shows the glutamate-gated currents in Xenopus oocytes that expressed GluR1(flip) both before and after application of 100 mM Cyclothiazide (CTZ) and/or toxin.

FIG. 7 shows the effects of recombinant Conikot-ikot peptides from the indicated species on glutamate-gated currents in Xenopus oocytes that express vertebrate GluR1(flip). Blue traces are the current response before toxin application. Red traces are one minute after toxin application.

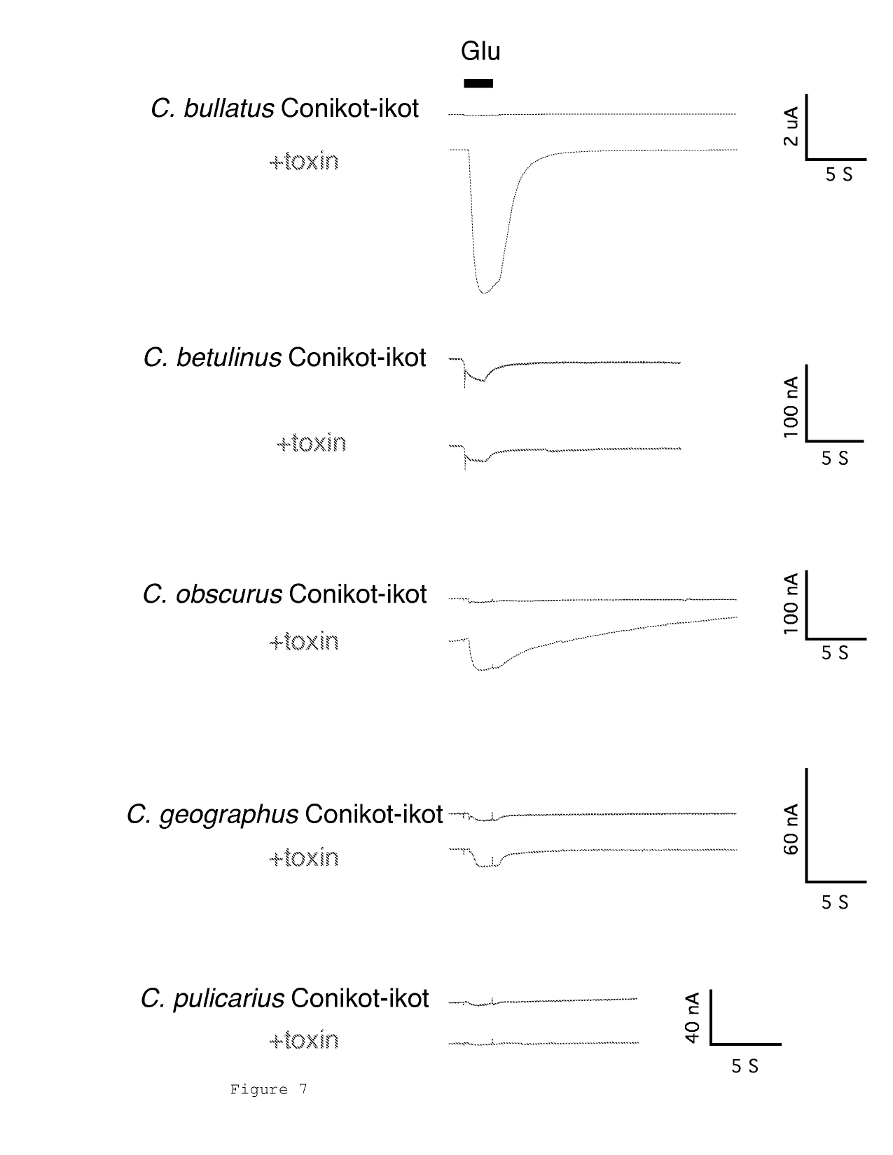

FIG. 8 shows effect of application of G12.2 on glutamate-gated currents in Xenopus oocytes expressing GluR2(flop), GluR4, or GluR3.

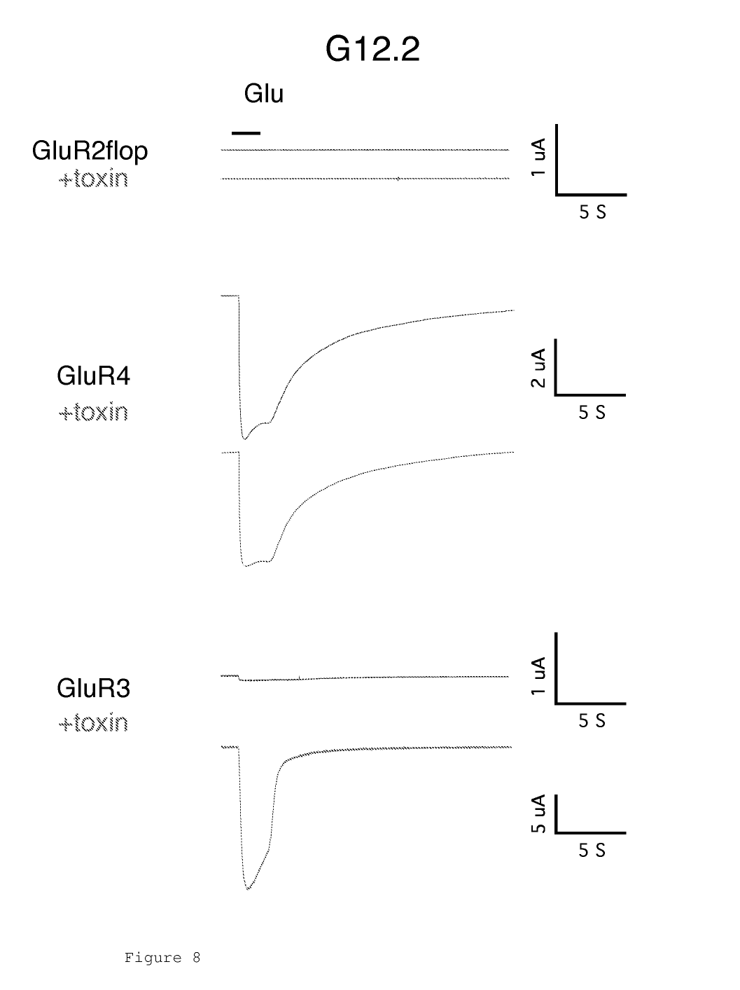

### DETAILED DESCRIPTION OF THE INVENTION

The invention relates to peptides (termed propeller peptides herein) and related peptides (termed peptides of the G12.2 family herein), about 60 to about 90 residues in length, which are naturally available in minute amounts in the venom of the cone snails or analogous to the naturally available peptides, and which blocks the desensitization of AMPA-type ionotropic glutamate receptors (AMPARs). More specifically, the present invention relates to the discovery and characterization of a toxin from the venomous fish-hunting cone snail, Conus striatus, which has a massive effect on GluR1 AMPAR-mediated current by inhibiting channel desensitization. The unprecedented biochemical properties of this toxin suggest that it is a molecular four-legged clamp which, when bound to the channel, locks it in an open state. The present invention also more specifically relates to the discovery of related toxins from other species of Conus.

This application presents the discovery and characterization of a novel class of peptides, which we designate propeller peptides, from the marine snails Conus striatus, Conus geographus, Conus bullatus, Conus obscurus, Conus pulicarius and others. The peptides were shown to be biologically active when injected into mice and goldfish. Activity on AMPARs is demonstrated. The application further presents the discovery and characterization of a class of related peptides, which are designated peptides of the G12.2 family, from various marine snails.

The propeller peptides of the present invention include those described above.

The peptides of the G12.2 family of the present invention include those described above.

The present invention is also directed to cDNA clones encoding the precursor of the biologically-active mature peptides and to the precursor peptides.

Desensitization in the continued presence of ligand is a conserved feature of all ligand-gated ion channels. Here we report that the venom of a predatory Conus marine snail contains a polypeptide toxin (con-ikot-ikot (propeller peptide)) that specifically blocks the desensitization of AMPA-type ionotropic glutamate receptors (AMPARs). Con-ikot-ikot is distinguished from previously described Conus toxins by its much larger size and in being the first known conopeptide targeted to AMPARs. Bacterially produced recombinant con-ikot-ikot had activity similar to that of native toxin with respect to its ability to enhance AMPAR-mediated glutamate-gated currents and its dramatic effects on fish and mice behavior. Interestingly, the stoichiometry of the active toxin appears reminiscent of the proposed subunit organization of AMPARs, i.e., a dimer of dimers. The biochemical characterization and functional effects of con-ikot-ikot suggest that it acts as a molecular four-legged clamp. Thus, once an AMPAR opens in response to agonist, the toxin apparently keeps the channel clamped in the open state. Presumably, each leg of the molecular clamp binds an individual AMPAR subunit in the open conformation and blocks the normal rapid transition of the receptor to a desensitized state. The discovery of con-ikot-ikot in a fish-hunting snail illustrates that receptor desensitization is of fundamental importance for nervous system function. We also report that the venom of predatory a Conus marine snail contains a polypeptide toxin (peptide of the G12.2 family) that has the same function of the con-ikot-ikot peptides, i.e., it also blocks desensitization of AMPARs.

The present invention, in another aspect, relates to a pharmaceutical composition comprising an effective amount of a propeller peptide, a mutein thereof, an analog thereof, an active fragment thereof or pharmaceutically acceptable salts or solvates. The present invention, in further aspect, relates to a pharmaceutical composition comprising an effective amount of a peptide of the G12.2 family, a mutein thereof, an analog thereof, an active fragment thereof or pharmaceutically acceptable salts or solvates. Such a pharmaceutical composition has the capability of acting at voltage-gated ion channels, ligand-gated ion channels and/or receptors, and are thus useful for treating a disorder or disease of a living animal body, including a human, which disorder or disease is responsive to the partial or complete blockade of such channels or receptors comprising the step of administering to such a living animal body, including a human, in need thereof a therapeutically effective amount of a pharmaceutical composition of the present invention. The propeller peptides and peptides of the G12.2 family are active at AMPARs and have utility for treating disorders which are associated with decreased or altered glutamatergic neurotransmission, such as Alzheimer's Disease.

Glutamate receptors, which mediate the majority of fast excitatory neurotransmission in the mammalian central nervous system (CNS), are activated by the excitatory amino acid, L-glutamate (for review see (25)).

Glutamate receptors can be divided into two distinct families. The G-protein or second messenger-linked “metabotropic” glutamate receptor family which can be subdivided into three groups (Group I, mGlu1 and mGlu5; Group II, mGlu2 and mGlu3; Group III, mGlu4, mGlu6, mGlu7, mGlu8) based on sequence homology and intracellular transduction mechanisms (for review see (26)). The “ionotropic” glutamate receptor family, which directly couple to ligand-gated cation channels, can be subdivided into at least three subtypes based on depolarizing activation by selective agonists, N-methyl-D-aspartate (NMDA), α-amino-3-hydroxy-5-methylisoxazole-4-propionic acid (AMPA) and kainic acid (KA) (for review see (7).

Native AMPA receptors (AMPAR) typically exist as heterotetramers consisting of combinations of four different protein subunits (GluR1-4) (for review see (27)). Receptor subunit diversity is increased further as each subunit can undergo alternative splicing of a 38 amino acid sequence in the extracellular region just before the fourth membrane spanning domain M4. Such editing results in so-called ‘flip’ and ‘flop’ receptor isoforms which differ in kinetic and pharmacological properties (28).

Additionally, post-transcriptional editing of GluR2 mRNA changes a neutral glutamine to a positively charged arginine within M2. In normal humans >99% GluR2 is edited in this way. AMPAR containing such edited GluR2 subunit exhibit low calcium permeability (29). There is a suggestion, however, that the number of AMPAR with high calcium permeability is elevated in certain disease-associated conditions (30).

AMPAR depolarization removes voltage dependent Mg2+ block of NMDA receptors which in turn leads to NMDA receptor activation, an integral stage in the induction of Long Term Potentiation (31). Long Term Potentiation is a physiological measure of increased synaptic strength following a repetitive stimulus or activity, such as occurs during learning.

Direct activation of glutamate receptors by agonists, in conditions where glutamate receptor function is reduced, increases the risk of excitotoxicity and additional neuronal damage. AMPAR positive allosteric modulators, alone, do not activate the receptor directly. However, when the ligand (L-glutamate or AMPA) is present AMPAR modulators increase receptor activity. Thus, AMPA receptor modulators only enhance synaptic function when glutamate is released and is able to bind at post-synaptic receptor sites.

Compounds which act as AMPAR positive allosteric modulators have been shown to increase ligand affinity for the receptor (32); reduce receptor desensitization and reduce receptor deactivation (33) Arai A C, Kessler M, Rogers G, Lynch G (2000) 58: 802-813) and facilitate the induction of LTP both in vitro (32) and in vivo (34). Such compounds also enhance the learning and performance of various cognitive tasks in rodent (35, 36), sub-human primate (37) and man (38).

It is envisaged that compounds that modulate glutamate receptor function may be useful in treating the following conditions and diseases: psychosis and psychotic disorders (including schizophrenia, schizo-affective disorder, schizophreniform diseases, brief reactive psychosis, child onset schizophrenia, “schizophrenia-spectrum” disorders such as schizoid or schizotypal personality disorders, acute psychosis, alcohol psychosis, drug-induced psychosis, autism, delerium, mania (including acute mania), manic depressive psychosis, hallucination, endogenous psychosis, organic psychosyndrome, paranoid and delusional disorders, puerperal psychosis, and psychosis associated with neurodegenerative diseases such as Alzheimer's disease); cognitive impairment (e.g. the treatment of impairment of cognitive functions including attention, orientation, memory (i.e. memory disorders, amnesia, amnesic disorders and age-associated memory impairment) and language function, and including cognitive impairment as a result of stroke, Alzheimer's disease, Aids-related dementia or other dementia states, as well as other acute or sub-acute conditions that may cause cognitive decline such as delirium or depression (pseudodementia states) trauma, aging, stroke, neurodegeneration, drug-induced states, neurotoxic agents), mild cognitive impairment, age related cognitive impairment, autism related cognitive impairment, Down's syndrome, cognitive deficit related to psychosis, post-electroconvulsive treatment related cognitive disorders; anxiety disorders (including generalised anxiety disorder, social anxiety disorder, agitation, tension, social or emotional withdrawal in psychotic patients, panic disorder, and obsessive compulsive disorder); neurodegenerative diseases (such as Alzheimer's disease, amyotrophic lateral sclerosis, motor neurone disease and other motor disorders such as Parkinson's disease (including relief from locomotor deficits and/or motor disability, including slowly increasing disability in purposeful movement, tremors, bradykinesia, hyperkinesia (moderate and severe), akinesia, rigidity, disturbance of balance and co-ordination, and a disturbance of posture), dementia in Parkinson's disease, dementia in Huntington's disease, neuroleptic-induced Parkinsonism and tardive dyskinesias, neurodegeneration following stroke, cardiac arrest, pulmonary bypass, traumatic brain injury, spinal cord injury or the like, and demyelinating diseases such as multiple sclerosis and amyotrophic lateral sclerosis); depression (which term includes bipolar (manic) depression (including type I and type II), unipolar depression, single or recurrent major depressive episodes with or without psychotic features, catatonic features, melancholic features, atypical features (e.g. lethargy, over-eating/obesity, hypersomnia) or postpartum onset, seasonal affective disorder and dysthymia, depression-related anxiety, psychotic depression, and depressive disorders resulting from a general medical condition including, but not limited to, myocardial infarction, diabetes, miscarriage or abortion); post-traumatic stress syndrome; attention deficit disorder; attention deficit hyperactivity disorder; drug-induced (phencyclidine, ketamine and other dissociative anaesthetics, amphetamine and other psychostimulants and cocaine) disorders; Huntingdon's chorea; tardive dyskinesia; dystonia; myoclonus; spasticity; obesity; stroke; sexual dysfunction; and sleep disorders. In addition, it is envisaged that compounds that modulate glutamate receptor function may be useful in treating non-impaired subjects for enhancing performance in sensory-motor and cognitive tasks and memory encoding.

The invention is further directed to the use of these peptides for screening drugs for activity at the receptor of these peptides and to isolate and assay receptors.

The peptides of the present invention are identified by isolation from Conus venom. Alternatively, the peptides of the present invention are identified using recombinant DNA techniques by screening cDNA libraries of various Conus species using conventional techniques such as the use of reverse-transcriptase polymerase chain reaction (RT-PCR) or the use of degenerate probes. Primers for RT-PCR are based on conserved sequences in the signal sequence and 3′ untranslated region of the propeller peptide genes. Clones which hybridize to these probes are analyzed to identify those which meet minimal size requirements, i.e., clones having approximately 300 nucleotides (for a precursor peptide), as determined using PCR primers which flank the cDNA cloning sites for the specific cDNA library being examined. These minimal-sized clones are then sequenced. The sequences are then examined for the presence of a peptide having the characteristics noted above for peptides. The biological activity of the peptides identified by this method is tested as described herein, in U.S. Pat. No. 5,635,347 or conventionally in the art.

These peptides are sufficiently small to be chemically synthesized by techniques well known in the art. The peptides are synthesized by a suitable method, such as by exclusively solid-phase techniques (Merrifield solid-phase synthesis), by partial solid-phase techniques, by fragment condensation or by classical solution couplings. Suitable techniques are exemplified by the disclosures of U.S. Pat. Nos. 4,105,603; 3,972,859; 3,842,067; 3,862,925; 4,447,356; 5,514,774; and 5,591,821, each incorporated herein.

Various ones of these propeller peptides and peptides of the G12.2 family can also be obtained by isolation and purification from specific Conus species using the techniques described in U.S. Pat. Nos. 4,447,356; 5,514,774 and 5,591,821, the disclosures of which are incorporated herein by reference.

The propeller peptides and peptides of the G12.2 family can also be produced by recombinant DNA techniques well known in the art. Such techniques are described by Sambrook and Russell (16). Production of recombinant proteins is described by, e.g., Gellissen (41), Bai and Nussinov (42) and Coligan et al. (43).

Muteins, analogs or active fragments, of the foregoing propeller peptides and peptides of the G12.2 family are also contemplated here. See, e.g., Hammerland et al. (17). Derivative muteins, analogs or active fragments of the propeller peptides and peptides of the G12.2 family may be synthesized according to known techniques, including conservative amino acid substitutions, such as outlined in U.S. Pat. No. 5,545,723 (see particularly col. 2, line 50 to col. 3, line 8); 5,534,615 (see particularly col. 19, line 45 to col. 22, line 33); and 5,364,769 (see particularly col. 4, line 55 to col. 7, line 26), each incorporated herein by reference.

Pharmaceutical compositions containing a compound, such as a peptide, of the present invention as the active ingredient can be prepared according to conventional pharmaceutical compounding techniques. See, for example, Remington: The Science and Practice of Pharmacy, 21st Ed., Lippincott Williams & Wilkins, Philadelphia, 2005. Typically, an antagonistic amount of active ingredient will be admixed with a pharmaceutically acceptable carrier. The carrier may take a wide variety of forms depending on the form of preparation desired for administration, e.g., intravenous, oral, parenteral or intrathecally. For examples of delivery methods see U.S. Pat. No. 5,844,077, incorporated herein by reference.

“Pharmaceutical composition” means physically discrete coherent portions suitable for medical administration. “Pharmaceutical composition in dosage unit form” means physically discrete coherent units suitable for medical administration, each containing a daily dose or a multiple (up to four times) or a sub-multiple (down to a fortieth) of a daily dose of the active compound in association with a carrier and/or enclosed within an envelope. Whether the composition contains a daily dose, or for example, a half, a third or a quarter of a daily dose, will depend on whether the pharmaceutical composition is to be administered once or, for example, twice, three times or four times a day, respectively.

The term “salt”, as used herein, denotes acidic and/or basic salts, formed with inorganic or organic acids and/or bases, preferably basic salts. While pharmaceutically acceptable salts are preferred, particularly when employing the compounds of the invention as medicaments, other salts find utility, for example, in processing these compounds, or where non-medicament-type uses are contemplated. Salts of these compounds may be prepared by art-recognized techniques.

Examples of such pharmaceutically acceptable salts include, but are not limited to, inorganic and organic addition salts, such as hydrochloride, sulphates, nitrates or phosphates and acetates, trifluoroacetates, propionates, succinates, benzoates, citrates, tartrates, fumarates, maleates, methane-sulfonates, isothionates, theophylline acetates, salicylates, respectively, or the like. Lower alkyl quaternary ammonium salts and the like are suitable, as well.

As used herein, the term “pharmaceutically acceptable” carrier means a non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations.

Wetting agents, emulsifiers and lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. Examples of pharmaceutically acceptable antioxidants include, but are not limited to, water soluble antioxidants such as ascorbic acid, cysteine hydrochloride, sodium bisulfite, sodium metabisulfite, sodium sulfite, and the like; oil soluble antioxidants, such as ascorbyl palmitate, butylated hydroxyanisole (BHA), butylated hydroxytoluene (BHT), lecithin, propyl gallate, alpha-tocopherol and the like; and the metal chelating agents such as citric acid, ethylenediamine tetraacetic acid (EDTA), sorbitol, tartaric acid, phosphoric acid and the like.

For oral administration, the compounds can be formulated into solid or liquid preparations such as capsules, pills, tablets, lozenges, melts, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents, and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques. The active agent can be encapsulated to make it stable to passage through the gastrointestinal tract while at the same time allowing for passage across the blood brain barrier. See for example, WO 96/11698.

For parenteral administration, the compound may be dissolved in a pharmaceutical carrier and administered as either a solution or a suspension. Illustrative of suitable carriers are water, saline, dextrose solutions, fructose solutions, ethanol, or oils of animal, vegetative or synthetic origin. The carrier may also contain other ingredients, for example, preservatives, suspending agents, solubilizing agents, buffers and the like. When the compounds are being administered intrathecally, they may also be dissolved in cerebrospinal fluid.

A variety of administration routes are available. The particular mode selected will depend of course, upon the particular drug selected, the severity of the disease state being treated and the dosage required for therapeutic efficacy. The methods of this invention, generally speaking, may be practiced using any mode of administration that is medically acceptable, meaning any mode that produces effective levels of the active compounds without causing clinically unacceptable adverse effects. Such modes of administration include oral, rectal, sublingual, topical, nasal, transdermal or parenteral routes. The term “parenteral” includes subcutaneous, intravenous, epidural, irrigation, intramuscular, release pumps, or infusion. For example, administration of the active agent according to this invention may be achieved using any suitable delivery means, including those described in U.S. Pat. No. 5,844,077, incorporated herein by reference.

Alternatively, targeting therapies may be used to deliver the active agent more specifically to certain types of cell, by the use of targeting systems such as antibodies or cell specific ligands. Targeting may be desirable for a variety of reasons, e.g. if the agent is unacceptably toxic, or if it would otherwise require too high a dosage, or if it would not otherwise be able to enter the target cells.

The active agents, which are peptides, can also be administered in a cell based delivery system in which a DNA sequence encoding an active agent is introduced into cells designed for implantation in the body of the patient, especially in the spinal cord region. Suitable delivery systems are described in U.S. Pat. No. 5,550,050 and published PCT Application Nos. WO 92/19195, WO 94/25503, WO 95/01203, WO 95/05452, WO 96/02286, WO 96/02646, WO 96/40871, WO 96/40959 and WO 97/12635. Suitable DNA sequences can be prepared synthetically for each active agent on the basis of the developed sequences and the known genetic code.

The active agent is preferably administered in a therapeutically effective amount. By a “therapeutically effective amount” or simply “effective amount” of an active compound is meant a sufficient amount of the compound to treat the desired condition at a reasonable benefit/risk ratio applicable to any medical treatment. The actual amount administered, and the rate and time-course of administration, will depend on the nature and severity of the condition being treated. Prescription of treatment, e.g. decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington: The Science and Practice of Pharmacy.

Dosage may be adjusted appropriately to achieve desired drug levels, locally or systemically. Typically the active agents of the present invention exhibit their effect at a dosage range from about 0.001 mg/kg to about 250 mg/kg, preferably from about 0.01 mg/kg to about 100 mg/kg of the active ingredient, more preferably from a bout 0.05 mg/kg to about 75 mg/kg. A suitable dose can be administered in multiple sub-doses per day. Typically, a dose or sub-dose may contain from about 0.1 mg to about 500 mg of the active ingredient per unit dosage form. A more preferred dosage will contain from about 0.5 mg to about 100 mg of active ingredient per unit dosage form. Dosages are generally initiated at lower levels and increased until desired effects are achieved. In the event that the response in a subject is insufficient at such doses, even higher doses (or effective higher doses by a different, more localized delivery route) may be employed to the extent that patient tolerance permits. Continuous dosing over, for example, 24 hours or multiple doses per day are contemplated to achieve appropriate systemic levels of compounds.

Advantageously, the compositions are formulated as dosage units, each unit being adapted to supply a fixed dose of active ingredients. Tablets, coated tablets, capsules, ampoules and suppositories are examples of dosage forms according to the invention.

It is only necessary that the active ingredient constitute an effective amount, i.e., such that a suitable effective dosage will be consistent with the dosage form employed in single or multiple unit doses. The exact individual dosages, as well as daily dosages, are determined according to standard medical principles under the direction of a physician or veterinarian for use humans or animals.

The pharmaceutical compositions will generally contain from about 0.0001 to 99 wt. %, preferably about 0.001 to 50 wt. %, more preferably about 0.01 to 10 wt. % of the active ingredient by weight of the total composition. In addition to the active agent, the pharmaceutical compositions and medicaments can also contain other pharmaceutically active compounds. Examples of other pharmaceutically active compounds include, but are not limited to, analgesic agents, cytokines and therapeutic agents in all of the major areas of clinical medicine. When used with other pharmaceutically active compounds, the peptides of the present invention may be delivered in the form of drug cocktails. A cocktail is a mixture of any one of the compounds useful with this invention with another drug or agent. In this embodiment, a common administration vehicle (e.g., pill, tablet, implant, pump, injectable solution, etc.) would contain both the instant composition in combination with a supplementary potentiating agent. The individual drugs of the cocktail are each administered in therapeutically effective amounts. A therapeutically effective amount will be determined by the parameters described above; but, in any event, is that amount which establishes a level of the drugs in the area of body where the drugs are required for a period of time which is effective in attaining the desired effects.

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., Molecular Cloning (Cold Spring Harbor Laboratory Press, Cold Spring Harbor, N.Y., 1982); Sambrook et al., Molecular Cloning, 2nd Ed. (Cold Spring Harbor Laboratory Press, Cold Spring Harbor, N.Y., 1989); Sambrook and Russell, Molecular Cloning, 3rd Ed. (Cold Spring Harbor Laboratory Press, Cold Spring Harbor, N.Y., 2001); Ausubel et al., Current Protocols in Molecular Biology (John Wiley & Sons, updated through 2008); Glover, DNA Cloning (IRL Press, Oxford, 1985); Anand, Techniques for the Analysis of Complex Genomes, (Academic Press, New York, 1992); Guthrie and Fink, Guide to Yeast Genetics and Molecular Biology (Academic Press, New York, 1991); Harlow and Lane, Antibodies, (Cold Spring Harbor Laboratory Press, Cold Spring Harbor, N.Y., 1998); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, (Blackwell Scientific Publications, Oxford, 1988); Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), 4th Ed., (Univ. of Oregon Press, Eugene, Oreg., 2000).

### EXAMPLES

The present invention can be described by reference to the following Examples, which are offered by way of illustration and are not intended to limit the invention in any manner. Standard techniques well known in the art or the techniques specifically described below were utilized.

### Example 1

### Methods for Examples 1-5

Biochemistry: 200 mg of crude venom isolated from C. striatus was purified by reverse phase HPLC using a binary buffer system (Buffer A: 0.1 M TFA. Buffer B: 0.1% TFA, 90% acetonitrile). Gradients were 5-65% Buffer B, 30 minutes, followed by additional separation at 65-100% Buffer B, 10 minutes. All purifications were performed using Vydac C18 columns: preparative columns (22 mm×25 cm, 15 μm particle size, 300 Å pore size), semi-preparative (10 mm×25 cm, 5 μm particle size, 300 Å pore) or analytical columns (4.6 mm×25 cm, 5 μm particle size, 300 Å pore size). Active fractions were further purified by two C18 separations from 23-45% B90 in 45 min and 25-40% B90 in 50 min. Purified peptide was sequenced by the University of Utah Core Facility using Edman N-terminal peptide sequencing. ESI/MS and MALDI TOF analysis was performed by the University of Utah Mass Spectrometry and Proteomics Core Facility. RNA was isolated from venom ducts by grinding with a mortar and pestle in ice cold RLT (RNeasy mini kit Qiagen). Primers designed to the con-ikot-ikot sequence were used to perform 5′ and 3′ RACE to identify the full cDNA sequence.

Recombinant con-ikot-ikot: cDNA encoding con-ikot-ikot was cloned in frame with thioredoxin pET32b+ (Novagen) (pCSW164). E. coli Rosetta-gami B (Novagen) containing pCSW164 were harvested 4 hrs after IPTG induction. Bacteria were lysed using an Avestin homoginizer in binding buffer (20 mM Phosphate 0.5 M NaCl, 20 mM imidazole pH7.4 and 1× Complete Protease Inhibitors (Roche)). FPLC purification of the protein fusion was performed using His-trap HP Nickel affinity column (GE healthcare). Recombinant protein was digested with enterokinase light chain (New England Biolabs; 2 mM Phosphate 50 mM NaCl 20 mM imidazole). The final purification was performed by reverse phase HPLC. Refolding experiments used a mixture of oxidized and reduced glutathione (1 mM GSSG, 2 mM GSH, 100 mM Tris, 10 mM EDTA pH7.5).

Protein Electrophoresis: Samples were prepared by adding purified native protein to RIPA buffer (1% Triton x-100, 0.5% DOC, 0.1% SDS, 0.15 M NaCl) with 50 mM Tris pH 7.5 or 50 mM Tris pH 7.5 and 1 M NaCl, or 0.01 M Na-acetate pH4. Samples were incubated at 37° C. 1 hour or 4° C. for pH 4 samples. Equal volume of sample buffer 6% SDS with or without DTT samples were then boiled 10 minutes before being separated on 15% precast SDS gel (Biorad)

Electrophysiological Studies: Xenopus oocytes were injected with cRNA prepared from p59/2, GluR1(flop); GluR1(flip) plasmid; pRB14, GluR2; pRB312, GluR3; pK46, GluR4; U08261, NR1; pNR2A251, NR2A; pDM1318, GluR1 flip-LIVBP D60-471. Oocyte electrophysiological recordings were performed as described previously (18).

Behavioral Analysis: Intracranial injections of propeller toxin were performed on 22-28 day old Swiss Webster mice (39, 40). Intramuscular injections of propeller were performed on goldfish Carassius auratus.

### Example 2

### Isolation of Propeller Peptide Con-ikot-ikot

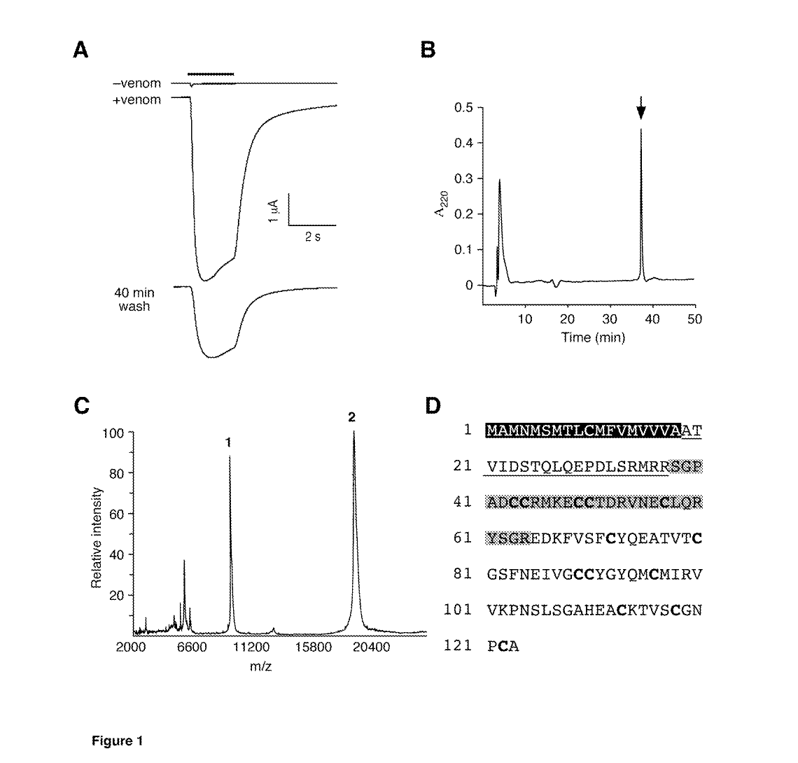

We screened a number of Conus venoms for activities that modified GluR1 AMPAR currents in Xenopus oocytes. In the absence of venom, we recorded small, rapidly desensitizing currents in response to bath application of 1 mM glutamate. In contrast, preincubation with venom from Conus striatus increased the size of the peak current by over an order of magnitude; presumably a consequence of the toxin-mediated decrease in the rate of receptor desensitization (FIG. 1a).

Following serial HPLC-fractionation of the venom, we identified a single peak (FIG. 1b) with biological activity identical to that of the original fraction (data not shown). MALDI mass spectrometry revealed two major peaks with average molecular weights of 9,443 and 18,868 kD (FIG. 1c), and electrospray mass spectrometry analysis revealed two peaks centered at 18864 kD (primary) and 9433 (secondary) (data not shown). Peptide sequencing revealed a 27 amino acid N-terminal sequence.

Degenerate oligonucleotides based on this sequence were used to identify a single cDNA from Conus striatus (The predicted coding sequence is in uppercase and the poly-adenylation signal is underlined.) encoding a predicted 123 amino acid peptide with an 18 amino acid signal sequence, a 19 amino acid propeptide region and an 86 amino acid mature peptide with 13 cysteines and a calculated molecular weight of 9432 kD. This weight is in close agreement with the electrospray estimate (FIG. 1d). We named this toxin con-ikot-ikot (ikot-ikot is a Filipino word that means spinning around or turning around, a reference to the swimming phenotype observed in fish injected with the toxin (see below).

cDNA sequence (SEQ ID NO: 6):
cggcttctaatacgactcactatagggcaagcagtggtaacaacgcagag
tacgcggggggacggcagaccagctggggaccagacagacgtcaaacagc
atcgcagtcaggtgtggagatcccaagacacccagaagaaggagacagaa
gagttatcgttcgtaacacaATGGCCATGAACATGTCGATGACACTCTGC
ATGTTTGTAATGGTCGTCGTGGCAGCCACTGTCATTGATTCCACTCAGTT
ACAAGAACCAGATCTCAGTCGCATGCGACGCAGCGGGCCTGCTGACTGTT
GCAGGATGAAAGAGTGTTGCACCGACAGAGTGAACGAGTCTCTACAGCGC
TATTCTGGCCGGGAAGATAAATTCGTTTCGTTTTGTTATCAGGAGGCCAC
AGTCACATGTGGATCTTTTAACGAAATCGTGGGCTGTTGCTATGGATATC
AAATGTGCATGATACGAGTTGTGAAACCGAACAGTCTAAGTGGGGCCCAT
GAGGCGTGCAAAACCGTTTCTTGTGGTAACCCTTGCGCTTGAggtgtcct
cgcgccacgtcacctgtgtacagcgccgtcaccagagccctgatctttat
gcccttatctgtctttttgctctttcactctctgaagtcttgaggtttgt
tccattcttgtcaatcatctcacgcgcatccaagtaaataaaggtgacgt
gacaaac
Precursor peptide sequence (SEQ ID NO: 7):
MAMNMSMTLCMFVMVVVAATVIDSTQLQEPDLSRMRRSGPADCCRMKECC
TDRVNECLQRYSGREDKFVSFCYQEATVTCGSFNEIVGCCYGYQMCMIRV
VKPNSLSGAHEACKTVSCGNPCA

### Example 3

### Confirmation of Con-ikot-ikot as Active Component

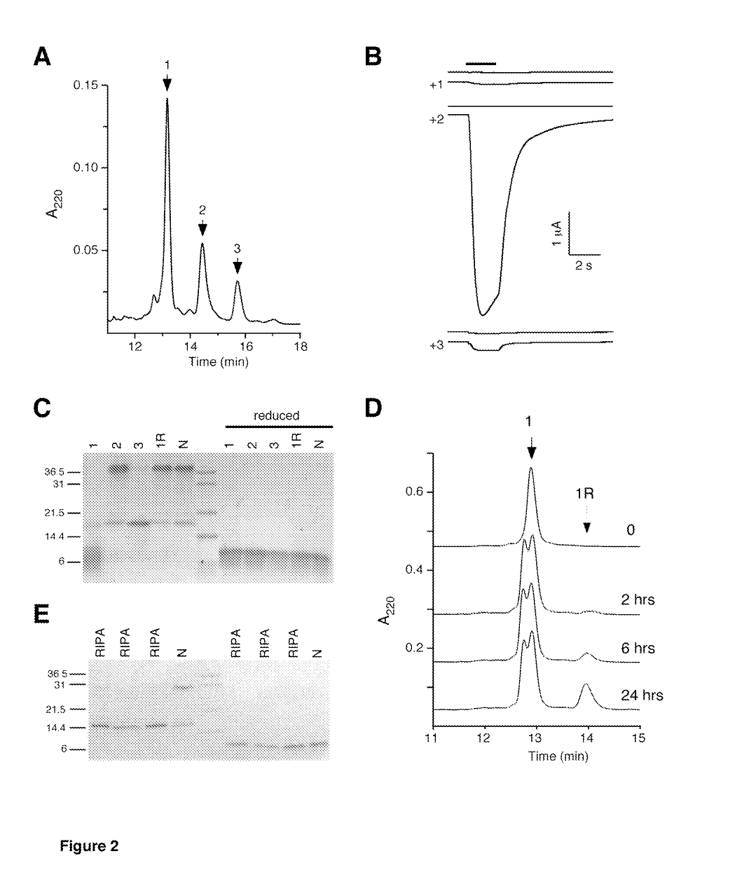

To establish that con-ikot-ikot was the active component of the venom, we expressed a 6×His-tagged thioreduxin-toxin fusion protein in bacteria. The recombinant protein was concentrated by Ni²⁺ column chromatography, treated with protease to release the toxin, and further fractionated by HPLC, which revealed a chromatogram consisting of 3 major peaks: peak 1 was the major component and first to elute, followed by minor peaks 2 and 3 (FIG. 2a). When tested for the ability to enhance glutamate-gated currents in oocytes, peaks 1 and 3 had no or little activity, whereas peak 2 greatly increased the current magnitude (FIG. 2b).

Analysis of the peaks and native toxin by gel electrophoreseis revealed 3 major bands (FIG. 2c). In the inactive peak 1 fraction, we observed a dominant band at approximately 9.5 kD (monomer), a minor band at roughly twice this size (19 kD), and no band at a position corresponding to four times the size (38 kD). In the inactive peak 3 fraction, we observed only a 19 kD band. In contrast, in the active peak 2 fraction, we observed a predominant 38 kD band as well as a minor 19 kD band. The bands observed in peak 2 were identical to that of native toxin.

Protein refolding experiments revealed that the inactive monomeric species could be converted to an active tetramer under conditions that promoted oxidation/reduction (1R) (FIG. 2c, d). All bands collapsed to the monomeric form under reducing conditions (FIG. 2c). Prolonged incubation of native toxin in RIPA buffer changed the ratio of bands from predominantly tetrameric to dimeric (FIG. 2e). These data, together with the mass spectrometry results (FIG. 1c), suggests that con-ikot-ikot forms a covalently linked dimer, presumably through an unpaired cysteine in the monomer, and that the mature toxin is a non-covalently linked dimer of dimers (FIG. 2c, e).

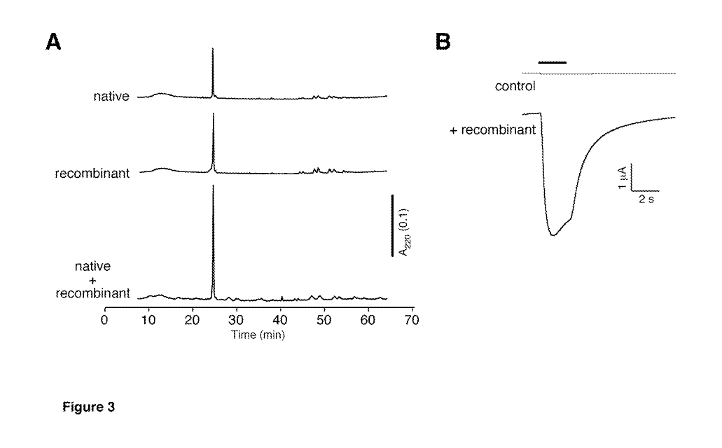

To verify that peak 2 recombinant con-ikot-ikot was equivalent to the native toxin, we showed that these peptides had identical co-elution profiles (FIG. 3a), molecular weight by MALDI (9433 and 18865), and biological activity (FIG. 3b).

### Example 4

### Pharmacological Specificity of Con-ikot-ikot

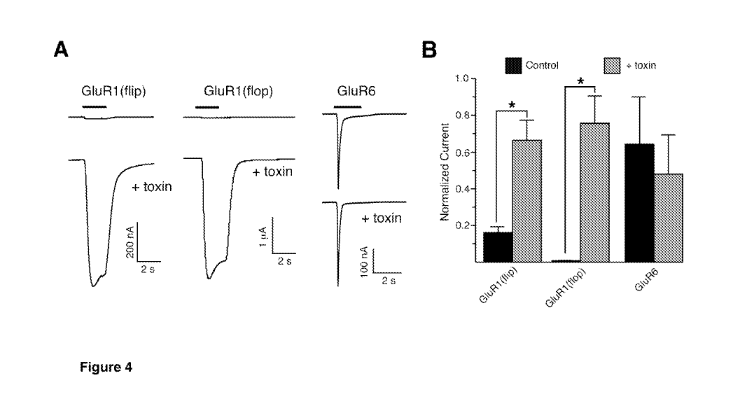

We assessed the pharmacological specificity of con-ikot-ikot by testing its ability to modify currents mediated by different AMPAR subunits as well as currents mediated by two other classes of iGluRs (7), i.e., those preferentially activated by either kainate or N-methyl-D-aspartic acid (NMDA). We found that con-ikot-ikot had no detectible activity on GluR6 kainate receptors or NR1/NR2A NMDA receptors (FIG. 4a, b). In contrast, we found that con-ikot-ikot differentially enhanced currents mediated by AMPAR subunits (FIG. 4a, b), with the largest enhancement observed with GluR1 (flop). The greater enhancement for flop vs. flip is the opposite of that observed for cyclothiazide, a modulator of desensitization (19). We observed no enhancement of the small GluR2 AMPAR-mediated currents (data not shown) and only minor enhancement of GluR4 AMPAR-mediated currents (FIG. 4a, b).

### Example 5

### Dose Response Study

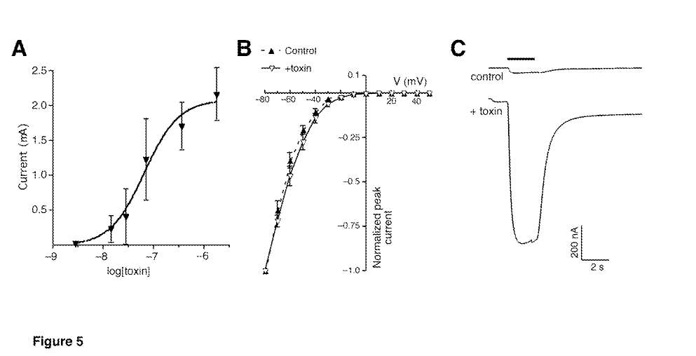

Evaluation of the dose-response relation for con-ikot-ikot revealed an apparent ECₛₒ of 67 nM (FIG. 5a). This value is almost certainly an underestimate because the relatively slow rate of perfusion in our oocyte recording chamber limits the detection of fast currents. In contrast to the marked effects on current desensitization, the current-voltage relation was not modified by the toxin, indicating that ion permeation was not altered by toxin (FIG. 5b). The extracellular portion of iGluRs consists of a large N-terminal domain and two smaller regions defined as 51 and S2, the sites of glutamate binding (20). Previous work has shown that recombinant receptors lacking the N-terminal domain (ΔNT) were functional and could be gated open by glutamate (21). To address the requirements for toxin modulation, we tested con-ikot-ikot on oocytes that expressed recombinant ΔNT-GluR1(flip). We found that the toxin enhanced glutamate-gated currents, indicating that the N-terminal domain was not crucial for con-ikot-ikot binding (FIG. 5c).

### Example 6

### Binding Study

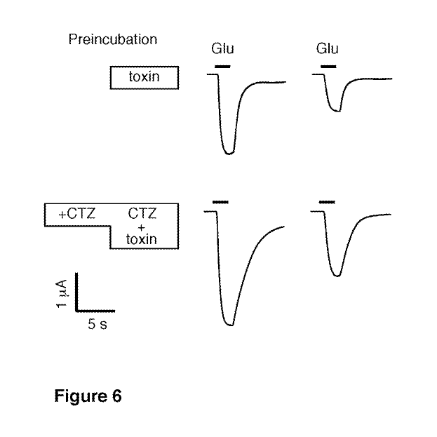

The non-specific drug cyclothiazide (CTZ) is a well known modulator of desensitization of AMPARs (22), causing block of desensitization of GluR1(flip) receptors (19). To address whether con-ikot-ikot and CTZ bound to a common site, we examined the effects of CTZ preincubation on con-ikot-ikot enhancement of GluR1(flip)-mediated current in Xenopus oocytes. We found that 100 μM CTZ blocked desensitization of GluR1(flip), but that the enhancement of current was rapidly washed out. In contrast, the effects of 6 μM con-ikot-ikot were long-lasting (FIG. 6). Preincubation of toxin-treated oocytes with CTZ did not decrease the acute or long-term effects of toxin, indicating that the two drugs bind to different sites (FIG. 6).

### Example 7

### Identification of Additional Propeller Peptides

We identified other propeller toxins in a variety of Conus species using a degenerate PCR approach. These additional toxins are set forth below. The predicted coding sequence is in uppercase and the poly-adenylation signal is underlined.

cDNA sequence (SEQ ID NO: 8) from Conus
geographus:
gctggggacgagacagacgtcaaacagcatcgcagtcaggtgtggagatc
ccaagacacccagaagaaggacacagaagagttatcgttcgtaatacaAT
GGCCATGAACATGTCGGTGATGCTCAGCGCGTTTGTAATGGTCGTCGTGT
CAGCCACTGTCACTGGTTTCACTCACTTACAAGAACCAGACCTCAGCCGC
ATGGAACGCAGCCCTCCCCCTCATAATGACTGTTGCAAAATGAAAGAGTG
TTGCGCCCAGACAACGGAGCTGTGTCTAAAGGAATTCCCTAACGAGGAAC
ATATCTATACTTCCACATGTTATCAGAGAGCCAGCCACGCATGTGGACAA
TTTAACGAAATCGTGGGCTGTTGCTATGGATACAGACAGTGCATGTTGCA
AAATGTGCAAAACTTGGGTCTTAACTGGGCCAACCAACAGTGCAAAGAGT
GGAATTGTTTGAACCCTTGCGAGTGAggtgtcctcacgccacgtcacctg
tgtacagcgccgtcaccagagacctgatccacgacactgtccctcccact
tcctcccactgtccctcccccttcctcccactgtccctccccctcccctt
cactgtcccttccccttcctcccactgtccctcccccttcctcccactgt
ccctcccctttctttctgctctgtcactccctgaagtcttgaggtctgtt
ccatccctatcattcatctcacttacatccaagtaaataaaggtgacgtg
ag
Precursor peptide sequence (SEQ ID NO: 9) from
Conus geographus:
MAMNMSVMLSAFVMVVVSATVTGFTHLQEPDLSRMERSPPPHNDCCKMKE
CCAQTTELCLKEFPNEEHIYTSTCYQRASHACGQFNEIVGCCYGYRQCML
QNVQNLGLNWANQQCKEWNCLNPCE
cDNA sequence (SEQ ID NO: 10) from Conus bullatus:
gcagacggcagaccagctggggaccagacagacgtcaaacagcatcgcag
tcaggtgtggagatcccaagacacccagaagaaggagaccagaagagtta
tcgttcgtaatacaATGGCCATGAACATGTCGATGACACTCTGCGTGTTT
GTAATGGTCGTCATGGCAGCCACTGTCATTGGTTCTACTCAGTTACAAGA
ACCAGACCTCGGTCGGACGGAACGCAGCCTGCCTGCTGACTGTTGCAACA
TGAAAAGCTGTTGCACCAAAAGCGTCTACACGTGTCTACAGGAATATCGT
GGCCGGGAAAATATCTTCGTTACGACTTGTTATCAGAGAGCCAGCGACAT
ATGTGGATCTTATAACGAAATCGTGGGCTGTTGCTATGGATATCAAATGT
GCATGATTCAAAATGTGAAACCGAACCAACTTCTTAACGCCCATGTTATG
TGCCGAAACACGGATTGTAATAACCCTTGCCAGTGAggtgtactcgcgcc
acgtcacctgtgtacagcgccgtcaccagagccctgatccacgacactgt
ccctccactttcctcccactctctctccccctccccccgcgccgttcaca
cacacacacacacacacacacacacacacacacacacacacacacacaca
cgtacacacacagacacacacacacacacacacacacacacacacacaca
tacacacacacacacacacacacacacacacatacacacgcgcgcacgcg
tgcgcactgtgtcccttcttaccactaatccccatctctccctctctcct
ctttatcccctcatctgtctttttgctctttcactctctgaagtcttgag
gtctgttccattcctgtcattcatctcactaacatccaagtaaataaagg
tgatgtgagaaactcttc
Precursor peptide sequence (SEQ ID NO: 11) from
Conus bullatus:
MAMNMSMTLCVFVMVVMAATVIGSTQLQEPDLGRTERSLPADCCNMKSCC
TKSVYTCLQEYRGRENIFVTTCYQRASDICGSYNEIVGCCYGYQMCMIQN
VKPNQLLNAHVMCRNTDCNNPCQ
cDNA sequence (SEQ ID NO: 12) from Conus obscurus:
gagacggcagaccagctggggaccagacagacgtcaaacagcatcgcagt
caggtgtggagatcccaaaacacccagaagaaggaaacagaagagttatc
gttcgtaatacaacggccATGAGCATGTCGATGACGTTCAGCGTGTTCGT
AATGGTCGTCATGGCAGCCACTATGATTGGTTCCACTCAGTTACGAGAAC
GAGGTCGAGGCCGCAGTCGCATGAAACGCCAGAACCGTGACTGTTGCATC
GCCTCCATTTACCAATGTCTGCAGCGATTTCCTGGACAAGAAAGTTATCG
CGCTCCGCCATGTCAAGGCGAAGCCGTCACCGAGTGTCCAAATACTGATA
TCGATAGCTGTTGCCCTGGATATACTACGTGCATGTCGGTACATGCGCAA
AATAACATTCGACCGGCTCATAACTTTTGTCAAAACAGGCTTTGTTATGG
TCCTTGAcgctgaggtgtcctcacgccacgtgaggtctgttccgtcccta
tcattcatctcacttacatccaagtaaataaaggtgacgcgagaaactct
t
Precursor peptide sequence (SEQ ID NO: 13) from
Conus obscurus:
MSMSMTFSVFVMVVMAATMIGSTQLRERGRGRSRMKRQNRDCCIASIYQC
LQRFPGQESYRAPPCQGEAVTECPNTDIDSCCPGYTTSMSVHAQNNIRPA
HNFCQNRLCYGP
cDNA sequence (SEQ ID NO: 14) from Conus
pulicarius:
tgacgtcaaacagcctcgcaggcaggtgtggagatcccaacacacccaga
agaaggaaacagaagagttatcgttcgtgatacaacggccATGAACATGT
GGATGACGTTCAGCGTGTGTGTAGTGGTTGTCATGGCAGTCACTGTCGCT
GGCTCCACTCCGTTACAAGAACAAGGCCTCAGCCTCGATGAACGCCGAAA
CCCTGGGTGCTGCCCCTGGGAATTCTATGACTGTCTGATAAAACGCGGGG
TGTGGAAGAACCGTTTGTATATTTGCTACAACATGGCTTCCAGGATTTGT
GCACCTGGCCGTTCTGGTGGTTGCTGCCCTATGCTTTTGAGCTGTTTTGA
AAGATGCAGGTCGAGGGACAGTGCAGTCTGCTATCACCGCTGCAAATTTG
CATCTTGCTGGATGTGAgaagacgccgactacagctgagatgtcgtcaca
gcacatcactcgtcacctcactggacacagcgccctgctccatgacgtca
ttcagcccccccccccttcctgccacacacacacacacacacacacacac
acacacacacagagtcttcctactgacccccacccttccatctctcctcc
tcacccttatctctcactctgttctttgacactctgtcttgacgtcagtt
ttactctttccacgtagttcattcccattcaagtcaataaagagtgatgt
gcg
Precursor peptide sequence (SEQ ID NO: 15) from
Conus pulicarius:
MNMWMTFSVCVVVVMAVTVAGSTPLQEQGLSLDERRNPGCCPWEFYDCLI
KRGVWKNRLYICYNMASRICAPGRSGGCCPMLLSCFERCRSRDSAVCYHR
CKFASCWM
cDNA sequence (SEQ ID NO: 31) from Conus bullatus
4_6_2:
GACCTCGGTCGGACGGAACGCAGCCTGCCTGCTGACTGTTGCAACATGAA
AAGCTGTTGCACCAAAAGCGTCTACACGTGTCTACAGGTGATCGTCGCCG
TGCTGCTCCTGACGGCCTGTCAGCTCATTACAGCTGATGACTCCAGAGGT
ACGCAGTTGCATCGTGCCCTGAGGAAGGCCACCAAACTCCCTGTGTCGAC
TCGCTGCATTACTCCAGGAACACGATGTAAGGTTCCGAGTCAATGCTGCA
GAGGTCCTTGCAAGAACGGTCGTTGTACTCCATCCCCTTCTGAATGGTAA
atgtggttgatccagcgcctgatcttcccccttctgtgctccatcctttt
ctgcctgagtcctccttacctgagagtggtcatgaaccactcatcaccta
ctcccctggaggcttcagaggagctacattgaaataaaagccgcattgca
aaaaaaaaaaaaaagcttgagctcgagtcctcgtc
Precursor peptide sequence (SEQ ID NO: 32) from
Conus bullatus 4_6_2s:
DLGRTERSLPADCCNMKSCCTKSVYTCLQVIVAVLLLTACQLITADDSRG
TQLHRALRKATKLPVSTRCITPGTRCKVPSQCCRGPCKNGRCTPSPSEW
cDNA sequence (SEQ ID NO: 33) from Conus
circumcisus:
GATCTCGGTCGCATGGGACGCAGCAGGGCTGGTGACTGTTGCAGGATGAA
AGAGTGTTGCACCGACAGAGTGAACACATGTCTACAGGGGTATTCTGGCC
GGGAAGATGTCTTCGTTTCGTTTTGTTATCAGGAGGCCACACACGCATGT
GGATCTTTTAACGAAATCGTGGGCTGTTGCTATGGATATCAAATGTGCAT
GATACAAGCTGTGAAACCGGGCCGTCTTAATCCGGCCCATGAGGCGTGCA
AAACCGTTGCTTGTGGTAAAAAAAAAAAAAAAAGCTTGAGCTCGAGTCCT
CGTC
Precursor peptide sequence (SEQ ID NO: 34) from
Conus circumcisus:
DLGRMGRSRAGDCCRMKECCTDRVNTCLQGYSGREDVFVSFCYQEATHAC
GSFNEIVGCCYGYQMCMIQAVKPGRLNPAHEACKTVACGKKKKKSLSSSP
R
cDNA sequence (SEQ ID NO: 35) from Conus
figulinus:
AAACGCAATGACCGCAGCAGCAGAAAATGTTGCGCCATAAAATCATACCT
GTGTCTTCAACATCATGGATGCCTTTCCCCGATTAATAGCAATTGTGCGG
AACAGTGCAAAACTACAGATACTAGCGGATGTGGATCAACTGTTGGTGAC
AACTGTTGCTCCGCTTACAAGTCCTGTCTGGTGGATTGCCAGATTTCACG
TGGAGATGAACATGGAGATCCACTCATTTCCTGTTATAATTATTGCGACC
AGCTGCACTCTTGTTAGtttgaaggagacgccgaccacagcggaggtgtg
ggcacgccgcatcactcgttacaacgccttggacacagccctgaaccatg
acgccgttcattccccaagtccagtcttcctgctcactccgatcactcct
tctctcctcctaacgcttctttctcatttctctatgacactctgaagtct
taatgtctgtcctaccattcttcattattctttccttatcaagtaaataa
agaatgatgtccgaaactcataaatcaaaaaaaaaaaaaaaaaaaaaaaa
aaaaaaaaaaaaaaaaaaaaaaaaaaaaggcttgagctcgagtcctcgtc
Precursor peptide sequence (SEQ ID NO: 36) from
Conus figulinus:
KRNDRSSRKCCAIKSYLCLQHHGCLSPINSNCAEQCKTTDTSGCGSTVGD
NCCSAYKSCLVDCQISRGDEHGDPLISCYNYCDQLHSC
cDNA sequence (SEQ ID NO: 37) from Conus
planorbis:
gcctgagccgcaagGACCTAAACATCCATAAATGCTGCATCCGGGAAACT
CATAGATGTGTCAGGCACTGCTGGGACCCGCACAATCCAGAGAGTGACTG
CGTGCTGGACTGCTACCACCGCGAGGCCTCCCACGTTTGTGGAACCACTG
GTGCTGGTGGCTGCTGCCCTGGGTTTGTGAACTGTTATGGACCGTGCACC
ATGGATGCTGATGCTAATTTAGATGTTTGCAGGCGCCGCTGCAAGCACGA
GTTTTGCTGGGACTCGTAAgacgttgactacagacgagatgtcgtcacgc
ctcatcacttgtaacaacactgtcgacagagcactgatgcgtgacgtcat
tcagctcccacttgcttcacccatcccataacaccccccccccccatcga
ccccttaccctgctcccagtcttcctactaaactctacccctccatcttt
cctcctctcccgtatctgtcgttttgctgttcgacaccctatcttgatgc
ctgtttcattctcccgttattccactcccattcaagtaaataaagagtaa
tgtgcaaaaaaaaaaaaaaaaaaaaaaaaaaaaagcttgagctcgagtcc
tcgtc
Precursor peptide sequence (SEQ ID NO: 38) from
Conus planorbis:
DLNIHKCCIRETHRCVRHCWDPHNPESDCVLDCYHREASHVCGTTGAGGC
CPGFVNCYGPCTMDADANLDVCRRRCKHEFCWDS
cDNA sequence (SEQ ID NO: 39) from Conus arenatus:
GACCTCAGCCTCGACGAACGCAATGAACCTATATGTTGCTCCTGGGAAAT
GCATGGCTGTCTTAATCAAAAAAGACGATGGCGGTTATTTTTGCTTATTT
GCTACAGAGAGGCTTCCATGATTTGTCCTGATGGCTGCTGCCCTGACGTT
TTGAGCTGTTTTCAAGAATGCATACCGTGGGACTATGATTGCTATGACAG
CTGCAGTTTTGTGGTTTGCTGAtcttgagaggatgccaactacacctgag
atggcgtcacgccacatcactcgtcacaacaccgtcacagcgcccatgac
gtcattcagccctttgacactctgtcttgacgtcagttttactctttcca
cgtagttcattcccattcaagtcaataaagagtgatgtgccaagctcaaa
aaaaaaaaaaaaaaaaaaaaaaaaaaaaaaaaaaaaaaaaaaagcttgag
ctcgagtcctcgtc
Precursor peptide sequence (SEQ ID NO: 40) from
Conus arenatus:
DLSLDERNEPICCSWEMHGCLNQKRRWRLFLLICYREASMICPDGCCPDV
LSCFQECIPWDYDCYDSCSFVVC
cDNA sequence (SEQ ID NO: 41) from Conus arenatus
1_1:
GGCCTCAGCCTCGATGAACGCAGTGTACCTGGATGCTGCCTCTGGGGCGT
TTATGACTGTCTGAGAGGAGGCGGGGGATGGAAGAGCCGTTTGTATTATT
GCTACTACGACAAGGCCTCCGCCTATTGTACAAGAGGCCGGCCTGGTGGC
TGCTGCCCTGGGCTTTTGCGATGTTTCGAAGACTGCACGTCGCCGGATAG
TGCAGTCTGCTTTGACCGCTGCAAATATGTGTCTTGCTGAtcttgagaag
acgccgactacagctgagatgtcgtcacagcacatcactcgtcacatcac
tcnacacagcgccctgctctatgacgtcattcagcccccgcctgcctcac
ccccccccccattactgacacacncacacacacacacacacacacagagt
cttcctactgaccccccacccttccatctctcctcctcacccttatctcc
cactctgttctttgacactctgtcttgacgtcaattttactcgttccacg
tagttcattcccattcaagtcnataaagantgangtgccnagctcnataa
aaaaanaaanaaaaaaaanaacgnntaaaccagnngngccnaatatnan
Precursor peptide sequence (SEQ ID NO: 42) from
Conus arenatus 1_1:
GLSLDERSVPGCCLWGVYDCLRGGGGWKSRLYYCYYDKASAYCTRGRPGG
CCPGLLRCFEDCTSPDSAVCFDRCKYVSC
Precursor peptide sequence (SEQ ID NO: 43) from
Conus betulinus:
DSTQLQEQKRNDRSSRKCCAVKSYLCLQGHGCLTPSDSSCAEQCKTTDTS
TCGSNAGDNCCSTYKSCLVDCQISRGNQHGDPLLICYNHCSQQRTYTGKW
KVGIRWS
cDNA sequence (SEQ ID NO: 44) from Conus virgo:
GAACGCAACAACGGCATCAGATGTTGCGCCATAGATTCATACCAATGTCT
TCGAGATAATGGATGCCTTTTCCCGATTGATATCGATTGTGCGGAACAGT
GCAAAACCACAGATACTAGCTATTGTGGACATGCTGCTGGTGACTGTTGC
TTCTCTTACAAGTCCTGTCTGGTGGATTGCCAGATTGAACGCGGAGATGA
ACCTGGAGATCAACTGCGTAACTGCTATAATAATTGCAACTCACAGGGTA
CGTACACAGGGAAGTGGAAAGTGGGGATACGTTGGTCCTGAgtggcatct
ttgtagtcgatggtacctctctccattacattgggtttcagaaaaaaaaa
aaaaaaaagcttgagctcgagtcctcgtc
Precursor peptide sequence (SEQ ID NO: 45) from
Conus virgo:
ERNNGIRCCAIDSYQCLRDNGCLFPIDIDCAEQCKTTDTSYCGHAAGDCC
FSYKSCLVDCQIERGDEPGDQLRNCYNNCNSQGTYTGKWKVGIRWS

### Example 8

### Isolation of Conus geographus 12.2

### A Member of the G12.2 Family

Identification of G12.2 HPLC purification: 500 mg of crude venom isolated from C. geographus was purified by reverse-phase HPLC via a binary buffer system (buffer A, 0.1% TFA; buffer B, 0.1% TFA, 90% acetonitrile). Gradients were 0%-100% buffer B in 50 min. All purifications were performed with Vydac C18 columns: preparative columns (22 mm×25 cm, 15° u particle size, 300 Å pore size), or semipreparative columns (10 mm×25 cm, 5 u particle size, 300 Å pore). Active fractions were further purified by two C18 separations from 10%-30% buffer B, 5 minutes followed by 30%-36% buffer B.

Sequence identification/mass: Purified peptide was sequenced by Edman N-terminal peptide sequencing by the University of Utah core facility MALDI TOF.

Recombinant protein expression and refolding: cDNA encoding G12.2 toxin cloned in frame with thioredoxin pET32b+ (Novagen) pCSW171. E. coli Rosettagami B containing pCSW171 was grown overnight 37 C, diluted 1:100. At 0.6 OD600, culture was cooled to 20 C, after 1 hour expression was induced with 0.2 mM IPTG for 4 hours. Bacteria were harvested and lysed by 3 passes at 12 Kpsi using Avestin homoginizer in Binding buffer (20 mM Phosphate 0.5M NaCl, 20 mM imidazole pH7.4 and 1× Complete protease inhibitors (Roche)). FPLC purification was performed using His-trap HP Nickle affinity column (GE healthcare). Recombinant protein was digested with Enterokinase light chain (New England Biolabs) (2 mM Phosphate 50 mM NaCl 20 mM imidazole) 24 hours at 20 C Refolding experiments were performed using mixture of oxidized and reduced glutathione (1 mM GSSG, 2 mM GSH, 100 mM Tris, 10 mM EDTA pH7.5).

cDNA sequence (SEQ ID NO: 46) from Conus
geographus:
aacgttgacgggcagtATGAACATTTACCTGTGTCTTGCTTTTCTTCTGT
TCCTGCCTTCTACCATAGTTGATTCAGGGCTTCTTGATAAAATTGAGACA
ATAAGGAATTGGAGACGTGATGAAAGCAAGTGTGATCGATGCAATTGCGC
CGAATTAAGATCATCCAGATGCACACAAGCTATCTTCTGCCTTACACCGG
AGTTATGCACACCGAGCATCTCATGTCCGACAGGTGAATGCCGCTGTACT
AAGTTCCATCAGTCAAGATGCACTAGATTCGTAGAATGCGTACCTAATAA
GTGTAGAGACGCATAGaggccagttccagcacatacagcaccatgatgcc
ctggacaatcgtgttgttggattgaatatgcccgtggcaggaatctgtcc
tacaaaaaa
Precursor peptide sequence (SEQ ID NO: 47) from
Conus geographus:
MNIYLCLAFLLFLPSTIVDSGLLDKIETIRNWRRDESKCDRCNCAELRSS
RCTQAIFCLTPELCTPSISCPTGECRCTKFHQSRCTRFVECVPNKCRDA

### Example 9

### Identification of Additional Peptides of Family G12.2

Expressed Sequence Tag analysis and PCR amplification: Method is as described in Walker et al. (44). First-strand synthesis of complementary DNA was primed from oligo(dT) extension at the PstI site of a linearized modified pUC13 plasmid using polyadenylated mRNA isolated from C. geographus venom ducts The products were size-fractionated by gel electrophoresis and used to transform Escherichia coli MC1061 to produce cDNA libraries (45). Expressed sequence tags were identified from single colonies randomly selected from Ampicillin-LB plates plated with Conus cDNA libraries (46). Insert sizes of the clones were analyzed by single colony PCR (47) with vector-specific oligonucleotides flanking the insert region (500 nM amount of each oligonucleotide, 2.5 mM MgCl2, 20 mM Tris-HCl, pH 8.4, 50 mM KCl, 250 g/ml bovine serum albumin, 125 M amount of each dNTP, and 0.5 unit of Taq DNA polymerase). Reaction mixtures were amplified (50 cycles of 25 s at 94° C., 25 s at 54° C., 2 min at 72° C.) using a 1605 Air Thermo-Cycler™ (Idaho Technology, Idaho Falls, Id.). Amplification products were analyzed by gel electrophoresis (1.5% agarose, 0.5 TBE buffer). Clones containing insert sizes larger than 400 base pairs in length were selected for sequencing. Templates were prepared using QIAprep Spin Miniprep kit (Qiagen, Valencia, Calif.) and submitted for fluorescent sequencing primed by oligonucleotides M13R and subsequently M13U at the Health Sciences Center Sequencing Facility, Eccles Institute of Human Genetics, University of Utah. Additional clones were identified by PCR amplification using DNA oligonucleotides directed to conserved regions of the cDNA sequence. All molecular biology techniques were as described by Sambrook et al., unless otherwise specified.

These additional toxins are set forth below. The predicted coding sequence is in uppercase.

cDNA sequence (SEQ ID NO: 48) from Conus
geographus 12.1:
agccttgatacagagctggtatctgctgttaatacttgaaagaacaagtg
ctgtgagccttcatctctctctgactttagtttgggtcctggagaaaacc
ttgacgggcagtATGAAAATTTACCTGTGTCTTGCTTTTGTTCTGCTCCT
GGCTTCTACCATAGTTGATTCAGGGCTTCTTGATAAAATTGAGACTATAA
GAAACTGGAAACGCGATGACAGCTATTGTGATGGATGCCTATGCACCATA
TTAAAAAAAGAGACTTGCACATCGACTATGAGCTGCAGGGGAACATGCCG
AAAAGAGTGGCCATGTTGGGAAGAAGACTGCTACTGTACTGAAATCCAAG
GTGGAGCTTGCGTCACACCCTCAGAATGCAAACCTGGAGAGTGTTGAgga
ttggagtggccagttccagcacatacagcaccatggtgccctggacaatc
gtctattgaattgaatatgcctgtggcaggaatctgtcctacaaaataaa
aaaatcataagttaaaaaa
Precursor peptide sequence (SEQ ID NO: 49) from
Conus geographus 12.1:
MKIYLCLAFVLLLASTIVDSGLLDKIETIRNWKRDDSYCDGCLCTILKKE
TCTSTMSCRGTCRKEWPCWEEDCYCTEIQGGACVTPSECKPGEC
cDNA sequence (SEQ ID NO: 50) from Conus erminius
12.1 3-3:
ctggtgtctgctgttaactcttgaaagaacaggtgtgctgtgagccttca
tctctctctgactttagtttgaggactggaggaaacgttgacaggcagtA
TGAAAATTTACCTGTGTCTTGCTATTCTTCTGCTTCTGGCTTCTACCATA
GTTGATTCAGGGCTTCTTGATATAACTCAGACTATAAGAAACTGGAGACG
CAAAGGAAGAGATGATAGCCAGTGTAATGAATGCAATTGCGCCCATTTAA
AAAAGGCCCAATGCACAGACCGTATCTACTGCGATTTTGAGGCACCTTGC
CCATCCGACTACTGGTGTCGGAACGGTAAATGCCTCTGTGCTCGTTTCCA
TATGGGAAGATGTTCTAAATCCTCAGAATGCATGCCTCATCAGTGTTGAg
gattggagtggccagttccagcacatacagcaccatggtcccctggacaa
ttgtgttgttgaattgaatctgcccgtggcaggaatctgtcctacaaaat
aaaaaatcataagttaaaaaaaaaaaaaaaaaaaaaaaa
Precursor peptide sequence (SEQ ID NO: 51) from
Conus erminius 12.1 3-3:
MKIYLCLAILLLLASTIVDSGLLDITQTIRNWRRKGRDDSQCNECNCAHL
KKAQCTDRIYCDFEAPCPSDYWCRNGKCLCARFHMGRCSKSSECMPHQC
cDNA sequence (SEQ ID NO: 52) from Conus
ventricosus 12.1:
ttgacgggcagtATGAAAATTTACCTGTGTCTTGCTGTTCTTCTGCTCCT
GGCTTCTACCATAGTTGATTCAGCGCTTCATCATAAAACTGAGGCTATAA
GAAACTGGAGACGCAAAGGAAGAGATGAAAACCAGTGTCCTGAATGCAGA
TGCTCCGAATTAACAACCGCCAGATGCGAAGAAAGTAAATACTGCCACCC
AGATGCCAGTTGCCCAACCAGCTCCTCCTGTAACAACGGTAAATGCCTCT
GTTCTCATTTCCTTGGTGGAAGATGCGTAAGCCACTCAGAATGCAATGAT
TCAGTCTGTTGAggattggagtgaccagttccagcacataaagcaccatg
gtcccctggacaatcgtgttgttgaattgaatctgcccgtggcaggagtc
tgtcctacaaaataaaaatcataagccaaaaaaaaaaaaaaaaaaaaaaa
aaaaaaaaaaaa
Precursor peptide sequence (SEQ ID NO: 53) from
Conus ventricosus 12.1:
MKIYLCLAVLLLLASTIVDSALHHKTEAIRNWRRKGRDENQCPECRCSEL
TTARCEESKYCHPDASCPTSSSCNNGKCLCSHFLGGRCVSHSECNDSVC
cDNA sequence (SEQ ID NO: 54) from Conus arenatus
12:
ATGAAAATTTACCTTTGTCTTGCCATTCTTCTGCTCCTGGCTTCTACCAT
AGTTTATTCAGCGCTTCTCGATAAAGCTGAGACTATAAGAAATTGGAGAC
GCAAAATCAGAGATGAAGCCCAATGTGAAGAATGCAGATGCTTAGAATTA
GTAATCCCGGAATGCACTGAAAGAAAATACTGCCACCCAGAGCACAGTTG
CCCAGTCAGACCCTACTGTAACAACGGTAAATGTCTGTGTAAACATTTCT
TTGGTGGAAGATGCGCAAAGCGCCCAGACTGCAATGATAGTCACTGTCGA
GCAGAGTAG
Precursor peptide sequence (SEQ ID NO: 55) from
Conus arenatus 12:
MKIYLCLAILLLLASTIVYSALLDKAETIRNWRRKIRDEAQCEECRCLEL
VIPECTERKYCHPEHSCPVRPYCNNGKCLCKHFFGGRCAKRPDCNDSHCR
AE
cDNA sequence (SEQ ID NO: 56) from Conus erminius
12.2:
CAAAGGAAGAGATGCAGGCCAGTGTACTGGTGGTACCTGCAGATGCTCCG
AACTAAAAGAAGCCATGTGCGCTGAAAGTAGATTATGCCTAAGCCCCAGT
TGCCCATCAAGCTCCGAATGTGCGGACAATCATTGCCTCTGTACTCATAT
CCTTCATGGAGTATGCGTAGACTCGACAGAATGCAACCCTAACAAGTGTT
GAggattggagtggccagttccagcacataaagcatcatggtctccttga
caatcgtgttgctgaaatgaatctacccatggcaggaatctgtcctacaa
aataaaaatcataagtaaagaaaaaaaaaaaaaaaaaaaaaaaaaaaaa
Precursor peptide sequence (SEQ ID NO: 57) from
Conus erminius 12.2:
KGRDAGQCTGGTCRCSELKEAMCAESRLCLSPSCPSSSECADNHCLCTHI
LHGVCVDSTECNPNKC
cDNA sequence (SEQ ID NO: 58) from Conus australis
26:
ATGGGCAGTATGAAAAAGTACCTGTGTCTTGCTATTCTTCTGCTTCTGGC
TTCTACCATAATTGATTCAGGGCCTTTTGATAAAATTGAGACTATAAGAA
AGTGGAGACGCGATGAAAGCCAGTGCGGTGACTGCAGATGCTTCGAGTTA
CGCACCGCCAGATGCACAGATAAACTTCAGTGCGAGTCACCcGACTTGTG
CAAACCCGTCGTCTCATGTGAGACTGGTAAATGCCACTGTACTCGTTTCA
GTTCGGGATGTTGCACTAGAACCGTGGAATGCATGCCTAAAAAGTGTTTC
TAA
Precursor peptide sequence (SEQ ID NO: 59) from
Conus australis 26:
MGSMKKYLCLAILLLLASTIIDSGPFDKIETIRKWRRDESQCGDCRCFEL
RTARCTDKLQCESPDLCKPVVSCETGKCHCTRFSSGCCTRTVECMPKKCF

Considering that con-ikot-ikot is a prominent component of Conus striatus venom it is likely to play an important role in the prey capture strategy of this fish-hunting cone snail. Conus striatus, one of the largest, most common species of piscivorous cone snails, is found from Hawaii to the Red Sea and East Africa. It aggressively stalks its fish prey, and can extend its proboscis at least 6 times the length of its shell. This allows large specimens of Conus striatus, which can reach up to 15 cm long, to strike their prey from a considerable distance. The immediate effect after the prey is struck is a rapid tetanic paralysis, resulting from a generalized excitotoxic shock. This is achieved by the massive depolarization of axons in the vicinity of the venom injection site. Conus peptides that keep Na⁺ channels in an open state and block K⁺ channels are key contributors to this generalized hyperexcitability (23). These venom components acting together are called “the lightning strike cabal”.

The discovery of con-ikot-ikots reveals a previously unrecognized mechanistic aspect to the lightning-strike cabal. Con-ikot-ikots presumably acts on glutamatergic circuitry, and in the peripheral nervous system of fish with the likely targeted circuitry in the lateral line. Depolarization of neurons combined with blockade of desensitization at synapses should cause an intense hyperexcitability leading to the rapid immobilization of the prey. Indeed, injection of 0.4 to 1 nM toxin into goldfish caused them to rotate around their longitudinal axis followed by death. Intracerebral injection of 0.04 nM toxin caused seizures in mice followed by death within minutes. To date, most modulators of AMPARs have been of limited therapeutic value. Con-ikot-ikots or soluble drugs modeled on con-ikot-ikot will have specific modulatory effects on synaptic transmission and thus contribute to the treatment of neurological disorders. Similarly, peptides of the G12.2 family or soluble drugs modeled on such peptides will have specific modulatory effects on synaptic transmission and thus contribute to the treatment of neurological disorders.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

It will be appreciated that the methods and compositions of the instant invention can be incorporated in the form of a variety of embodiments, only a few of which are disclosed herein. Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### BIBLIOGRAPHY

1. Terlau, H., and Olivera, B. M. (2004) Physiol. Rev. 84, 41-68.
2. Craig, A. G. et al. (1999) J. Biol. Chem. 274, 13752-13759.
3. Olivera, B. M. et al. (1985) Toxicon 23, 277-282.
4. Maillo, M. et al. (2002) Toxicon 40, 401-407.
5. Cruz, L. J. et al. (1987) J. Biol. Chem. 262, 15821-15824.
6. Jimenez, E. C. et al. (1996) J. Biol. Chem. 281, 28002-28005.
7. Dingledine, R. et al. (1999). The glutamate receptor ion channels. Pharmacol Rev 51, 7-61.
8. Mayer, M. L. (2006). Glutamate receptors at atomic resolution. Nature 440, 456-462.
9. Hansen, K. B. et al. (2007). Structural aspects of AMPA receptor activation, desensitization and deactivation. Curr Opin Neurobiol 17, 281-288.
10. Sun, Y. et al. (2002). Mechanism of glutamate receptor desensitization. Nature 417, 245-253.
11. Nicoll, R. A. et al. (2006). Auxiliary subunits assist AMPA-type glutamate receptors. Science 311, 1253-1256.
12. Zheng, Y. et al. (2004). SOL-1 is a CUB-domain protein required for GLR-1 glutamate receptor function in C. elegans. Nature 427, 451-457 (2004).
13. Walker, C. S. et al. Reconstitution of invertebrate glutamate receptor function depends on stargazin-like proteins. Proc Natl Acad Sci USA 103, 10781-6 (2006).
14. Walker, C. S. et al. Conserved SOL-1 proteins regulate ionotropic glutamate receptor desensitization. Proc Natl Acad Sci USA 103, 10787-92 (2006).
15. Van de Steen, P. et al. (1998) Critical Rev. in Biochem. and Mol. Biol. 33, 151-208.
16. Sambrook and Russell (2001) Molecular Cloning, 3rd Ed., Cold Spring Harbor Laboratory, Cold Spring Harbor, N.Y.
17. Hammerland, L. G. et al. (1992). Eur. J. Pharmacol. 226:239-244.
18. Strutz-Seebohm, N. et al. Functional analysis of Caenorhabditis elegans glutamate receptor subunits by domain transplantation. J Biol Chem 278, 44691-701 (2003).
19. Partin, K. M. et al. (1996). AMPA receptor flip/flop mutants affecting deactivation, desensitization, and modulation by cyclothiazide, aniracetam, and thiocyanate. J Neurosci 16, 6634-6647.
20. Armstrong, N. et al. (1998). Structure of a glutamate-receptor ligand-binding core in complex with kainate. Nature 395, 913-917.
21. Pasternack, A. et al. (2002). Alpha-amino-3-hydroxy-5-methyl-4-isoxazolepro-pionic acid (AMPA) receptor channels lacking the N-terminal domain. J Biol Chem 277, 49662-49667.
22. Arai, A. C. and Kessler, M. (2007). Pharmacology of ampakine modulators: from AMPA receptors to synapses and behavior. Curr Drug Targets 8, 583-602.
23. Terlau, H. et al. (1996). Strategy for rapid immobilization of prey by a fish-hunting marine snail. Nature 381, 148-151.
24. McIntosh, J. M. et al. (1998) Methods Enzymol 294, 605-624.
25. Watkins, J. C. et al. (1990) Trends Pharmacol Sci 11: 25-33.
26. Conn, P. J. and Pinn, J. P. (1997) Ann Rev Pharmacol Toxicol 37: 205-237.
27. Bettler B and Muller C (1995) Neuropharmacology 34: 123-139.
28. Sommer, B. Et al. (1990) Science 249: 1580-1585.
29. Burnachev, N. et al. (1992) Neuron 8: 189-198.
30. Weiss, J. H. and Sensi, S. L. (2000) Trends in Neurosci 23: 365-371.
31. Bliss, T. V. P. and Collingridge, G. L. (1993) Nature 361: 31-9.
32. Arai, A. et al. (1996) Neuroreport 7: 2211-5.
33. Arai, A. C. et al. (2000) Mol Pharmacol 58: 802-813.
34. Staubli, U. et al. (1994) Proc Natl Acad Sci USA 91: 11158-11162.
35. Zivkovic, I. et al. (1995) J Pharm Exp Ther 272: 300-309.
36. Lebrun, C. et al. (2000) Eu J Pharmacol 401: 205-212.
37. Thompson, D. M. et al. (1995) Proc Natl Acad Sci USA 92: 7667-7671.
38. Ingvar, M. et al. (1997) Exp Neurol 146: 553-559.
39. Clark, C. et al. (1981). A toxin from the venom of the marine snail Conus geographus which acts on the vertebrate central nervous system. Toxicon 19:691-699.
40. Lirazan, M. B. et al. (2000). The spasmodic peptide defines a new conotoxin superfamily. Biochem 39:1583-1588.
41. Gellissen, G., Ed. (2005). Production of Recombinant Proteins: Novel Microbial and Eukaryotic Expression Systems, John Wiley & Sons, Inc., N.Y.
42. Bai, Y. and Nussinov, R., Eds. (2006). Protein Folding Protocols, Methods Mol Biol 350, Springer.
43. Coligan, J. E. et al., Eds. (2007). Current Protocols in Protein Science, John Wiley and Sons, Inc. (updated through 2008).
44. Walker, C. S. et al. (1999). The T-Superfamily of Conotoxins. J Biol Chem 274, 30664-71.
45. Colledge, C. J. et al. (1992) Toxicon 30, 1111-1116.
46. Kohn, A. J. et al. (1991) Science 252, 1651-1656.
47. Dorfman, D. M. (1989) BioTechniques 7, 568-570.

What is claimed is:

1. An isolated peptide selected from the group consisting of
(a) SGPADCCRMKECCTDRVNECLQRYSGREDKFVSFCYQEATVTCGSF NEIVGCCYGYQMCMIRVVKPNSLSGAHEACKTVSCGNPCA (SEQ ID NO:1; con-ikot-ikot);
(b) SPPPHNDCCKMKECCAQTTELCLKEFPNEEHIYTSTCYQRASHACGQ FNEIVGCCYGYRQCMLQNVQNLGLNWANQQCKEWNCLNPCE (SEQ ID NO:2);
(c) SLPADCCNMKSCCTKSVYTCLQEYRGRENIFVTTCYQRASDICGSY NEIVGCCYGYQMCMIQNVKPNQLLNAHVMCRNTDCNNPCQ (SEQ ID NO:3);
(d) QNRDCCIASIYQCLQRFPGQESYRAPPCQGEAVTECPNTDIDSCCPGY TTCMSVHAQNNIRPAHNFCQNRLCYGP (SEQ ID NO:4);
(e) NPGCCPWEFYDCLIKRGVWKNRLYICYNMASRICAPGRSGGCCPM LLSCFERCRSRDSAVCYHRCKFASCWM (SEQ ID NO:5);
(f) SLPADCCNMKSCCTKSVYTCLQVIVAVLLLTACQLITADDSRGTQLH RALRKATKLPVSTRCITPGTRCKVPSQCCRGPCKNGRCTPSPSEW (SEQ ID NO:16);
(g) SRAGDCCRMKECCTDRVNTCLQGYSGREDVFVSFCYQEATHACGSF NEIVGCCYGYQMCMIQAVKPGRLNPAHEACKTVACGKKKKKSLSSSPR (SEQ ID NO:17);
(h) KRNDRSSRKCCAIKSYLCLQHHGCLSPINSNCAEQCKTTDTSGCGST VGDNCCSAYKSCLVDCQISRGDEHGDPLISCYNYCDQLHSC (SEQ ID NO:18);
(i) DLNIHKCCIRETHRCVRHCWDPHNPESDCVLDCYHREASHVCGTTG AGGCCPGFVNCYGPCTMDADANLDVCRRRCKHEFCWDS (SEQ ID NO:19);
(j) DLSLDERNEPICCSWEMHGCLNQKRRWRLFLLICYREASMICPDGCC PDVLSCFQECIPWDYDCYDSCSFVVC (SEQ ID NO:20);
(k) GLSLDERSVPGCCLWGVYDCLRGGGGWKSRLYYCYYDKASAYC TRGRPGGCCPGLLRCFEDCTSPDSAVCFDRCKYVSC (SEQ ID NO:21);
(l) NDRSSRKCCAVKSYLCLQGHGCLTPSDSSCAEQCKTTDTSTCGSNA GDNCCSTYKSCLVDCQISRGNQHGDPLLICYNHCSQQRTYTGKWKVGIRWS (SEQ ID NO:22);
(m) NNGIRCCAIDSYQCLRDNGCLFPIDIDCAEQCKTTDTSYCGHAAGDC CFSYKSCLVDCQIERGDEPGDQLRNCYNNCNSQGTYTGKWKVGIRWS (SEQ ID NO:23);
(n) DESKCDRCNCAELRSSRCTQAIFCLTPELCTPSISCPTGECRCTKFHQS RCTRFVECVPNKCRDA (SEQ ID NO:24);
(o)
DDSYCDGCLCTILKKETCTSTMSCRGTCRKEWPCWEEDCYCTEIQGG ACVTPSECKPGEC (SEQ ID NO:25);
(p) DDSQCNECNCAHLKKAQCTDRIYCDFEAPCPSDYWCRNGKCLCARF HMGRCSKSSECMPHQC (SEQ ID NO:26);
(q) DENQCPECRCSELTTARCEESKYCHPDASCPTSSSCNNGKCLCSHFL GGRCVSHSECNDSVC (SEQ ID NO:27);
(r) DEAQCEECRCLELVIPECTERKYCHPEHSCPVRPYCNNGKCLCKHFF GGRCAKRPDCNDSHCRAE (SEQ ID NO:28);
(s) DAGQCTGGTCRCSELKEAMCAESRLCLSPSCPSSSECADNHCLCTHI LHGVCVDSTECNPNKC (SEQ ID NO:29); and
(t) DESQCGDCRCFELRTARCTDKLQCESPDLCKPVVSCETGKCHCTRF SSGCCTRTVECMPKKCF (SEQ ID NO:30).
(u) a derivative of (a), (b), (c), (d), (e), (f), (g), (h), (i), (j), (k), (l), (m), (n), (o), (p), (q), (r), (s) or (t), wherein the derivative is the peptide (a), (b), (c), (d) or (e) in which the Pro residues may be substituted with hydroxyl-Pro; the Arg residues may be substituted by Lys, ornithine, homoargine, nor-Lys, N-methyl-Lys, N,N-dimethyl-Lys, N,N,N-trimethyl-Lys or any synthetic basic amino acid; the Lys residues may be substituted by Arg, ornithine, homoargine, nor-Lys, or any synthetic basic amino acid; the Tyr residues may be substituted with any synthetic hydroxy containing amino acid; the Ser residues may be substituted with Thr or any synthetic hydroxylated amino acid; the Thr residues may be substituted with Ser or any synthetic hydroxylated amino acid; the Phe and Trp residues may be substituted with any synthetic aromatic amino acid; and the Asn, Ser, Thr or Hyp residues may be glycosylated; the Tyr residues may also be substituted with the 3-hydroxyl or 2-hydroxyl isomers (meta-Tyr or ortho-Tyr, respectively) and corresponding O-sulpho- and O-phospho-derivatives or may be substituted with nor-Tyr, nitro-Tyr, mono-iodo-Tyr or di-iodo-Tyr; the aliphatic amino acids may be substituted by synthetic derivatives bearing non-natural aliphatic branched or linear side chains CₙH₂ₙ₊₂ up to and including n=8; the Leu residues may be substituted with Leu(D); the Trp residues may be substituted with halo-Trp, Trp(D) or halo-Trp(D); the halogen is iodo, chloro, fluoro or bromo which is optionally radiolabeled.

2. The isolated peptide of claim 1, wherein the peptide is selected from the group consisting of SEQ ID NO:1, SEQ ID NO:2, SEQ ID NO:3, SEQ ID NO:4, SEQ ID NO:5, SEQ ID NO:16, SEQ ID NO:17, SEQ ID NO:18, SEQ ID NO:19, SEQ ID NO:20, SEQ ID NO:21, SEQ ID NO:22 and SEQ ID NO:23.

3. The isolated peptide of claim 1, wherein the peptide is selected from the group consisting of SEQ ID NO:24, SEQ ID NO:25, SEQ ID NO:26, SEQ ID NO:27, SEQ ID NO:28, SEQ ID NO:29 and SEQ ID NO:30.

4. The isolated peptide of claim 1, which is modified to contain an O-glycan, an S-glycan or an N-glycan.

5. A pharmaceutical composition comprising a peptide of claim 1 or a pharmaceutically acceptable salt or solvate thereof and a pharmaceutically acceptable carrier.

6. A pharmaceutical composition comprising a peptide of claim 2 or a pharmaceutically acceptable salt or solvate thereof and a pharmaceutically acceptable carrier.

7. A pharmaceutical composition comprising a peptide of claim 3 or a pharmaceutically acceptable salt or solvate thereof and a pharmaceutically acceptable carrier.

8. A pharmaceutical composition comprising a peptide of claim 4 or a pharmaceutically acceptable salt or solvate thereof and a pharmaceutically acceptable carrier.

9. A method of identifying compounds that mimic the therapeutic activity of a propeller peptide or a peptide of the G12.2 family, comprising the steps of: (a) conducting a biological assay on a test compound to determine the therapeutic activity; and (b) comparing the results obtained from the biological assay of the test compound to the results obtained from the biological assay of a peptide of claim 1.

10. An isolated nucleic acid which encodes a peptide of claim 1.

11. An isolated nucleic acid which encodes a precursor peptide of a peptide of claim 1.

12. The isolated nucleic acid of claim 11 selected from the group consisting of SEQ ID NO:6, SEQ ID NO:8, SEQ ID NO:10, SEQ ID NO:12, SEQ ID NO:14, SEQ ID NO:31, SEQ ID NO:33, SEQ ID NO:350, SEQ ID NO:37, SEQ ID NO:39, SEQ ID NO:41, SEQ ID NO:44, SEQ ID NO:46, SEQ ID NO:48, SEQ ID NO:50, SEQ ID NO:52, SEQ ID NO:54, SEQ ID NO:56 and SEQ ID NO:58.

13. A mature peptide derived from the precursor encoded by the nucleic acid sequence of claim 12.

14. A precursor peptide selected from the group consisting of SEQ ID NO:7, SEQ ID NO:9, SEQ ID NO:11, SEQ ID NO:13, SEQ ID NO:15, SEQ ID NO:32, SEQ ID NO:34, SEQ ID NO:36, SEQ ID NO:38, SEQ ID NO:40, SEQ ID NO:42, SEQ ID NO:43, SEQ ID NO:45, SEQ ID NO:47, SEQ ID NO:49, SEQ ID NO:51, SEQ ID NO:53, SEQ ID NO:55, SEQ ID NO:57 and SEQ ID NO:59.


SEQUENCE LISTING (59 sequences)

SEQ ID NO: 1 - protein, 86 aa, Conus striatus
SGPADCCRMKECCTDRVNECLQRYSGREDKFVSFCYQEATVTCGSFNEIVGCCYGYQMCMIRVVKPNSLSGAHEACKTVSCGNPCA

SEQ ID NO: 2 - protein, 88 aa, Conus geographus
SPPPHNDCCKMKECCAQTTELCLKEFPNEEHIYTSTCYQRASHACGQFNEIVGCCYGYRQCMLQNVQNLGLNWANQQCKEWNCLNPCE

SEQ ID NO: 3 - protein, 86 aa, Conus bullatus
SLPADCCNMKSCCTKSVYTCLQEYRGRENIFVTTCYQRASDICGSYNEIVGCCYGYQMCMIQNVKPNQLLNAHVMCRNTDCNNPCQ

SEQ ID NO: 4 - protein, 75 aa, Conus obscurus
QNRDCCIASIYQCLQRFPGQESYRAPPCQGEAVTECPNTDIDSCCPGYTTCMSVHAQNNIRPAHNFCQNRLCYGP

SEQ ID NO: 5 - protein, 72 aa, Conus pulicarius
NPGCCPWEFYDCLIKRGVWKNRLYICYNMASRICAPGRSGGCCPMLLSCFERCRSRDSAVCYHRCKFASCWM

SEQ ID NO: 6 - DNA, 707 bp, Conus striatus
cggcttctaatacgactcactatagggcaagcagtggtaacaacgcagagtacgcggggggacggcagaccagctggggaccagacagacgtcaaacagcatcgcagtcaggtgtggagatcccaagacacccagaagaaggagacagaagagttatcgttcgtaacacaatggccatgaacatgtcgatgacactctgcatgtttgtaatggtcgtcgtggcagccactgtcattgattccactcagttacaagaaccagatctcagtcgcatgcgacgcagcgggcctgctgactgttgcaggatgaaagagtgttgcaccgacagagtgaacgagtgtctacagcgctattctggccgggaagataaattcgtttcgttttgttatcaggaggccacagtcacatgtggatcttttaacgaaatcgtgggctgttgctatggatatcaaatgtgcatgatacgagttgtgaaaccgaacagtctaagtggggcccatgaggcgtgcaaaaccgtttcttgtggtaacccttgcgcttgaggtgtcctcgcgccacgtcacctgtgtacagcgccgtcaccagagccctgatctttatgcccttatctgtctttttgctctttcactctctgaagtcttgaggtttgttccattcttgtcaatcatctcacgcgcatccaagtaaataaaggtgacgtgacaaac

SEQ ID NO: 7 - protein, 123 aa, Conus striatus
MAMNMSMTLCMFVMVVVAATVIDSTQLQEPDLSRMRRSGPADCCRMKECCTDRVNECLQRYSGREDKFVSFCYQEATVTCGSFNEIVGCCYGYQMCMIRVVKPNSLSGAHEACKTVSCGNPCA

SEQ ID NO: 8 - DNA, 752 bp, Conus geographus
gctggggacgagacagacgtcaaacagcatcgcagtcaggtgtggagatcccaagacacccagaagaaggacacagaagagttatcgttcgtaatacaatggccatgaacatgtcggtgatgctcagcgcgtttgtaatggtcgtcgtgtcagccactgtcactggtttcactcacttacaagaaccagacctcagccgcatggaacgcagccctccccctcataatgactgttgcaaaatgaaagagtgttgcgcccagacaacggagctgtgtctaaaggaattccctaacgaggaacatatctatacttccacatgttatcagagagccagccacgcatgtggacaatttaacgaaatcgtgggctgttgctatggatacagacagtgcatgttgcaaaatgtgcaaaacttgggtcttaactgggccaaccaacagtgcaaagagtggaattgtttgaacccttgcgagtgaggtgtcctcacgccacgtcacctgtgtacagcgccgtcaccagagacctgatccacgacactgtccctcccacttcctcccactgtccctcccccttcctcccactgtccctccccctccccttcactgtcccttccccttcctcccactgtccctcccccttcctcccactgtccctcccctttctttctgctctgtcactccctgaagtcttgaggtctgttccatccctatcattcatctcacttacatccaagtaaataaaggtgacgtgag

SEQ ID NO: 9 - protein, 125 aa, Conus geographus
MAMNMSVMLSAFVMVVVSATVTGFTHLQEPDLSRMERSPPPHNDCCKMKECCAQTTELCLKEFPNEEHIYTSTCYQRASHACGQFNEIVGCCYGYRQCMLQNVQNLGLNWANQQCKEWNCLNPCE

SEQ ID NO: 10 - DNA, 918 bp, Conus bullatus
gcagacggcagaccagctggggaccagacagacgtcaaacagcatcgcagtcaggtgtggagatcccaagacacccagaagaaggagaccagaagagttatcgttcgtaatacaatggccatgaacatgtcgatgacactctgcgtgtttgtaatggtcgtcatggcagccactgtcattggttctactcagttacaagaaccagacctcggtcggacggaacgcagcctgcctgctgactgttgcaacatgaaaagctgttgcaccaaaagcgtctacacgtgtctacaggaatatcgtggccgggaaaatatcttcgttacgacttgttatcagagagccagcgacatatgtggatcttataacgaaatcgtgggctgttgctatggatatcaaatgtgcatgattcaaaatgtgaaaccgaaccaacttcttaacgcccatgttatgtgccgaaacacggattgtaataacccttgccagtgaggtgtactcgcgccacgtcacctgtgtacagcgccgtcaccagagccctgatccacgacactgtccctccactttcctcccactctctctccccctccccccgcgccgttcacacacacacacacacacacacacacacacacacacacacacacacacacacacgtacacacacagacacacacacacacacacacacacacacacacacacatacacacacacacacacacacacacacacacatacacacgcgcgcacgcgtgcgcactgtgtcccttcttaccactaatccccatctctccctctctcctctttatcccctcatctgtctttttgctctttcactctctgaagtcttgaggtctgttccattcctgtcattcatctcactaacatccaagtaaataaaggtgatgtgagaaactcttc

SEQ ID NO: 11 - protein, 123 aa, Conus bullatus
MAMNMSMTLCVFVMVVMAATVIGSTQLQEPDLGRTERSLPADCCNMKSCCTKSVYTCLQEYRGRENIFVTTCYQRASDICGSYNEIVGCCYGYQMCMIQNVKPNQLLNAHVMCRNTDCNNPCQ

SEQ ID NO: 12 - DNA, 551 bp, Conus obscurus
gagacggcagaccagctggggaccagacagacgtcaaacagcatcgcagtcaggtgtggagatcccaaaacacccagaagaaggaaacagaagagttatcgttcgtaatacaacggccatgagcatgtcgatgacgttcagcgtgttcgtaatggtcgtcatggcagccactatgattggttccactcagttacgagaacgaggtcgaggccgcagtcgcatgaaacgccagaaccgtgactgttgcatcgcctccatttaccaatgtctgcagcgatttcctggacaagaaagttatcgcgctccgccatgtcaaggcgaagccgtcaccgagtgtccaaatactgatatcgatagctgttgccctggatatactacgtgcatgtcggtacatgcgcaaaataacattcgaccggctcataacttttgtcaaaacaggctttgttatggtccttgacgctgaggtgtcctcacgccacgtgaggtctgttccgtccctatcattcatctcacttacatccaagtaaataaaggtgacgcgagaaactctt

SEQ ID NO: 13 - protein, 112 aa, Conus obscurus
MSMSMTFSVFVMVVMAATMIGSTQLRERGRGRSRMKRQNRDCCIASIYQCLQRFPGQESYRAPPCQGEAVTECPNTDIDSCCPGYTTCMSVHAQNNIRPAHNFCQNRLCYGP

SEQ ID NO: 14 - DNA, 703 bp, Conus pulicarius
tgacgtcaaacagcctcgcaggcaggtgtggagatcccaacacacccagaagaaggaaacagaagagttatcgttcgtgatacaacggccatgaacatgtggatgacgttcagcgtgtgtgtagtggttgtcatggcagtcactgtcgctggctccactccgttacaagaacaaggcctcagcctcgatgaacgccgaaaccctgggtgctgcccctgggaattctatgactgtctgataaaacgcggggtgtggaagaaccgtttgtatatttgctacaacatggcttccaggatttgtgcacctggccgttctggtggttgctgccctatgcttttgagctgttttgaaagatgcaggtcgagggacagtgcagtctgctatcaccgctgcaaatttgcatcttgctggatgtgagaagacgccgactacagctgagatgtcgtcacagcacatcactcgtcacctcactggacacagcgccctgctccatgacgtcattcagcccccccccccttcctgccacacacacacacacacacacacacacacacacacacagagtcttcctactgacccccacccttccatctctcctcctcacccttatctctcactctgttctttgacactctgtcttgacgtcagttttactctttccacgtagttcattcccattcaagtcaataaagagtgatgtgcg

SEQ ID NO: 15 - protein, 108 aa, Conus pulicarius
MNMWMTFSVCVVVVMAVTVAGSTPLQEQGLSLDERRNPGCCPWEFYDCLIKRGVWKNRLYICYNMASRICAPGRSGGCCPMLLSCFERCRSRDSAVCYHRCKFASCWM

SEQ ID NO: 16 - protein, 92 aa, Conus bullatus
SLPADCCNMKSCCTKSVYTCLQVIVAVLLLTACQLITADDSRGTQLHRALRKATKLPVSTRCITPGTRCKVPSQCCRGPCKNGRCTPSPSEW

SEQ ID NO: 17 - protein, 94 aa, Conus circumcisus
SRAGDCCRMKECCTDRVNTCLQGYSGREDVFVSFCYQEATHACGSFNEIVGCCYGYQMCMIQAVKPGRLNPAHEACKTVACGKKKKKSLSSSPR

SEQ ID NO: 18 - protein, 88 aa, Conus figulinus
KRNDRSSRKCCAIKSYLCLQHHGCLSPINSNCAEQCKTTDTSGCGSTVGDNCCSAYKSCLVDCQISRGDEHGDPLISCYNYCDQLHSC

SEQ ID NO: 19 - protein, 84 aa, Conus planorbis
DLNIHKCCIRETHRCVRHCWDPHNPESDCVLDCYHREASHVCGTTGAGGCCPGFVNCYGPCTMDADANLDVCRRRCKHEFCWDS

SEQ ID NO: 20 - protein, 73 aa, Conus arenatus
DLSLDERNEPICCSWEMHGCLNQKRRWRLFLLICYREASMICPDGCCPDVLSCFQECIPWDYDCYDSCSFVVC

SEQ ID NO: 21 - protein, 79 aa, Conus arenatus
GLSLDERSVPGCCLWGVYDCLRGGGGWKSRLYYCYYDKASAYCTRGRPGGCCPGLLRCFEDCTSPDSAVCFDRCKYVSC

SEQ ID NO: 22 - protein, 97 aa, Conus betulinus
NDRSSRKCCAVKSYLCLQGHGCLTPSDSSCAEQCKTTDTSTCGSNAGDNCCSTYKSCLVDCQISRGNQHGDPLLICYNHCSQQRTYTGKWKVGIRWS

SEQ ID NO: 23 - protein, 94 aa, Conus virgo
NNGIRCCAIDSYQCLRDNGCLFPIDIDCAEQCKTTDTSYCGHAAGDCCFSYKSCLVDCQIERGDEPGDQLRNCYNNCNSQGTYTGKWKVGIRWS

SEQ ID NO: 24 - protein, 65 aa, Conus geographus
DESKCDRCNCAELRSSRCTQAIFCLTPELCTPSISCPTGECRCTKFHQSRCTRFVECVPNKCRDA

SEQ ID NO: 25 - protein, 60 aa, Conus geographus
DDSYCDGCLCTILKKETCTSTMSCRGTCRKEWPCWEEDCYCTEIQGGACVTPSECKPGEC

SEQ ID NO: 26 - protein, 62 aa, Conus erminius
DDSQCNECNCAHLKKAQCTDRIYCDFEAPCPSDYWCRNGKCLCARFHMGRCSKSSECMPHQC

SEQ ID NO: 27 - protein, 62 aa, Conus ventricosus
DENQCPECRCSELTTARCEESKYCHPDASCPTSSSCNNGKCLCSHFLGGRCVSHSECNDSVC

SEQ ID NO: 28 - protein, 65 aa, Conus arenatus
DEAQCEECRCLELVIPECTERKYCHPEHSCPVRPYCNNGKCLCKHFFGGRCAKRPDCNDSHCRAE

SEQ ID NO: 29 - protein, 63 aa, Conus erminius
DAGQCTGGTCRCSELKEAMCAESRLCLSPSCPSSSECADNHCLCTHILHGVCVDSTECNPNKC

SEQ ID NO: 30 - protein, 63 aa, Conus australis
DESQCGDCRCFELRTARCTDKLQCESPDLCKPVVSCETGKCHCTRFSSGCCTRTVECMPKKCF

SEQ ID NO: 31 - DNA, 485 bp, Conus bullatus
gacctcggtcggacggaacgcagcctgcctgctgactgttgcaacatgaaaagctgttgcaccaaaagcgtctacacgtgtctacaggtgatcgtcgccgtgctgctcctgacggcctgtcagctcattacagctgatgactccagaggtacgcagttgcatcgtgccctgaggaaggccaccaaactccctgtgtcgactcgctgcattactccaggaacacgatgtaaggttccgagtcaatgctgcagaggtccttgcaagaacggtcgttgtactccatccccttctgaatggtaaatgtggttgatccagcgcctgatcttcccccttctgtgctccatccttttctgcctgagtcctccttacctgagagtggtcatgaaccactcatcacctactcccctggaggcttcagaggagctacattgaaataaaagccgcattgcaaaaaaaaaaaaaaagcttgagctcgagtcctcgtc

SEQ ID NO: 32 - protein, 99 aa, Conus bullatus
DLGRTERSLPADCCNMKSCCTKSVYTCLQVIVAVLLLTACQLITADDSRGTQLHRALRKATKLPVSTRCITPGTRCKVPSQCCRGPCKNGRCTPSPSEW

SEQ ID NO: 33 - DNA, 304 bp, Conus circumcisus
gatctcggtcgcatgggacgcagcagggctggtgactgttgcaggatgaaagagtgttgcaccgacagagtgaacacatgtctacaggggtattctggccgggaagatgtcttcgtttcgttttgttatcaggaggccacacacgcatgtggatcttttaacgaaatcgtgggctgttgctatggatatcaaatgtgcatgatacaagctgtgaaaccgggccgtcttaatccggcccatgaggcgtgcaaaaccgttgcttgtggtaaaaaaaaaaaaaaaagcttgagctcgagtcctcgtc

SEQ ID NO: 34 - protein, 101 aa, Conus circumcisus
DLGRMGRSRAGDCCRMKECCTDRVNTCLQGYSGREDVFVSFCYQEATHACGSFNEIVGCCYGYQMCMIQAVKPGRLNPAHEACKTVACGKKKKKSLSSSPR

SEQ ID NO: 35 - DNA, 599 bp, Conus figulinus
aacgcaatgaccgcagcagcagaaaatgttgcgccataaaatcatacctgtgtcttcaacatcatggatgcctttccccgattaatagcaattgtgcggaacagtgcaaaactacagatactagcggatgtggatcaactgttggtgacaactgttgctccgcttacaagtcctgtctggtggattgccagatttcacgtggagatgaacatggagatccactcatttcctgttataattattgcgaccagctgcactcttgttagtttgaaggagacgccgaccacagcggaggtgtgggcacgccgcatcactcgttacaacgccttggacacagccctgaaccatgacgccgttcattccccaagtccagtcttcctgctcactccgatcactccttctctcctcctaacgcttctttctcatttctctatgacactctgaagtcttaatgtctgtcctaccattcttcattattctttccttatcaagtaaataaagaatgatgtccgaaactcataaatcaaaaaaaaaaaaaaaaaaaaaaaaaaaaaaaaaaaaaaaaaaaaaaaaaaaaggcttgagctcgagtcctcgtc

SEQ ID NO: 36 - protein, 88 aa, Conus figulinus; identical to SEQ ID NO: 18

SEQ ID NO: 37 - DNA, 605 bp, Conus planorbis
gcctgagccgcaaggacctaaacatccataaatgctgcatccgggaaactcatagatgtgtcaggcactgctgggacccgcacaatccagagagtgactgcgtgctggactgctaccaccgcgaggcctcccacgtttgtggaaccactggtgctggtggctgctgccctgggtttgtgaactgttatggaccgtgcaccatggatgctgatgctaatttagatgtttgcaggcgccgctgcaagcacgagttttgctgggactcgtaagacgttgactacagacgagatgtcgtcacgcctcatcacttgtaacaacactgtcgacagagcactgatgcgtgacgtcattcagctcccacttgcttcacccatcccataacaccccccccccccatcgaccccttaccctgctcccagtcttcctactaaactctacccctccatctttcctcctctcccgtatctgtcgttttgctgttcgacaccctatcttgatgcctgtttcattctcccgttattccactcccattcaagtaaataaagagtaatgtgcaaaaaaaaaaaaaaaaaaaaaaaaaaaaagcttgagctcgagtcctcgtc

SEQ ID NO: 38 - protein, 84 aa, Conus planorbis; identical to SEQ ID NO: 19

SEQ ID NO: 39 - DNA, 464 bp, Conus arenatus
gacctcagcctcgacgaacgcaatgaacctatatgttgctcctgggaaatgcatggctgtcttaatcaaaaaagacgatggcggttatttttgcttatttgctacagagaggcttccatgatttgtcctgatggctgctgccctgacgttttgagctgttttcaagaatgcataccgtgggactatgattgctatgacagctgcagttttgtggtttgctgatcttgagaggatgccaactacacctgagatggcgtcacgccacatcactcgtcacaacaccgtcacagcgcccatgacgtcattcagccctttgacactctgtcttgacgtcagttttactctttccacgtagttcattcccattcaagtcaataaagagtgatgtgccaagctcaaaaaaaaaaaaaaaaaaaaaaaaaaaaaaaaaaaaaaaaaaaaaagcttgagctcgagtcctcgtc

SEQ ID NO: 40 - protein, 73 aa, Conus arenatus; identical to SEQ ID NO: 20

SEQ ID NO: 41 - DNA, 599 bp, Conus arenatus
  misc_feature (303)..(303): n is a, c, g, or t
ggcctcagcctcgatgaacgcagtgtacctggatgctgcctctggggcgtttatgactgtctgagaggaggcgggggatggaagagccgtttgtattattgctactacgacaaggcctccgcctattgtacaagaggccggcctggtggctgctgccctgggcttttgcgatgtttcgaagactgcacgtcgccggatagtgcagtctgctttgaccgctgcaaatatgtgtcttgctgatcttgagaagacgccgactacagctgagatgtcgtcacagcacatcactcgtcacatcactcnacacagcgccctgctctatgacgtcattcagcccccgcctgcctcacccccccccccattactgacacacncacacacacacacacacacacagagtcttcctactgaccccccacccttccatctctcctcctcacccttatctcccactctgttctttgacactctgtcttgacgtcaattttactcgttccacgtagttcattcccattcaagtcnataaagantgangtgccnagctcnataaaaaaanaaanaaaaaaaanaacgnntaaaccagnngngccnaatatnan

SEQ ID NO: 42 - protein, 79 aa, Conus arenatus; identical to SEQ ID NO: 21

SEQ ID NO: 43 - protein, 107 aa, Conus betulinus
DSTQLQEQKRNDRSSRKCCAVKSYLCLQGHGCLTPSDSSCAEQCKTTDTSTCGSNAGDNCCSTYKSCLVDCQISRGNQHGDPLLICYNHCSQQRTYTGKWKVGIRWS

SEQ ID NO: 44 - DNA, 379 bp, Conus virgo
gaacgcaacaacggcatcagatgttgcgccatagattcataccaatgtcttcgagataatggatgccttttcccgattgatatcgattgtgcggaacagtgcaaaaccacagatactagctattgtggacatgctgctggtgactgttgcttctcttacaagtcctgtctggtggattgccagattgaacgcggagatgaacctggagatcaactgcgtaactgctataataattgcaactcacagggtacgtacacagggaagtggaaagtggggatacgttggtcctgagtggcatctttgtagtcgatggtacctctctccattacattgggtttcagaaaaaaaaaaaaaaaaagcttgagctcgagtcctcgtc

SEQ ID NO: 45 - protein, 96 aa, Conus virgo
ERNNGIRCCAIDSYQCLRDNGCLFPIDIDCAEQCKTTDTSYCGHAAGDCCFSYKSCLVDCQIERGDEPGDQLRNCYNNCNSQGTYTGKWKVGIRWS

SEQ ID NO: 46 - DNA, 409 bp, Conus geographus
aacgttgacgggcagtatgaacatttacctgtgtcttgcttttcttctgttcctgccttctaccatagttgattcagggcttcttgataaaattgagacaataaggaattggagacgtgatgaaagcaagtgtgatcgatgcaattgcgccgaattaagatcatccagatgcacacaagctatcttctgccttacaccggagttatgcacaccgagcatctcatgtccgacaggtgaatgccgctgtactaagttccatcagtcaagatgcactagattcgtagaatgcgtacctaataagtgtagagacgcatagaggccagttccagcacatacagcaccatgatgccctggacaatcgtgttgttggattgaatatgcccgtggcaggaatctgtcctacaaaaaa

SEQ ID NO: 47 - protein, 99 aa, Conus geographus
MNIYLCLAFLLFLPSTIVDSGLLDKIETIRNWRRDESKCDRCNCAELRSSRCTQAIFCLTPELCTPSISCPTGECRCTKFHQSRCTRFVECVPNKCRDA

SEQ ID NO: 48 - DNA, 519 bp, Conus geographus
agccttgatacagagctggtatctgctgttaatacttgaaagaacaagtgctgtgagccttcatctctctctgactttagtttgggtcctggagaaaaccttgacgggcagtatgaaaatttacctgtgtcttgcttttgttctgctcctggcttctaccatagttgattcagggcttcttgataaaattgagactataagaaactggaaacgcgatgacagctattgtgatggatgcctatgcaccatattaaaaaaagagacttgcacatcgactatgagctgcaggggaacatgccgaaaagagtggccatgttgggaagaagactgctactgtactgaaatccaaggtggagcttgcgtcacaccctcagaatgcaaacctggagagtgttgaggattggagtggccagttccagcacatacagcaccatggtgccctggacaatcgtctattgaattgaatatgcctgtggcaggaatctgtcctacaaaataaaaaaatcataagttaaaaaa

SEQ ID NO: 49 - protein, 94 aa, Conus geographus
MKIYLCLAFVLLLASTIVDSGLLDKIETIRNWKRDDSYCDGCLCTILKKETCTSTMSCRGTCRKEWPCWEEDCYCTEIQGGACVTPSECKPGEC

SEQ ID NO: 50 - DNA, 539 bp, Conus ermineus
ctggtgtctgctgttaactcttgaaagaacaggtgtgctgtgagccttcatctctctctgactttagtttgaggactggaggaaacgttgacaggcagtatgaaaatttacctgtgtcttgctattcttctgcttctggcttctaccatagttgattcagggcttcttgatataactcagactataagaaactggagacgcaaaggaagagatgatagccagtgtaatgaatgcaattgcgcccatttaaaaaaggcccaatgcacagaccgtatctactgcgattttgaggcaccttgcccatccgactactggtgtcggaacggtaaatgcctctgtgctcgtttccatatgggaagatgttctaaatcctcagaatgcatgcctcatcagtgttgaggattggagtggccagttccagcacatacagcaccatggtcccctggacaattgtgttgttgaattgaatctgcccgtggcaggaatctgtcctacaaaataaaaaatcataagttaaaaaaaaaaaaaaaaaaaaaaaa

SEQ ID NO: 51 - protein, 99 aa, Conus ermineus
MKIYLCLAILLLLASTIVDSGLLDITQTIRNWRRKGRDDSQCNECNCAHLKKAQCTDRIYCDFEAPCPSDYWCRNGKCLCARFHMGRCSKSSECMPHQC

SEQ ID NO: 52 - DNA, 462 bp, Conus ventricosus
ttgacgggcagtatgaaaatttacctgtgtcttgctgttcttctgctcctggcttctaccatagttgattcagcgcttcatcataaaactgaggctataagaaactggagacgcaaaggaagagatgaaaaccagtgtcctgaatgcagatgctccgaattaacaaccgccagatgcgaagaaagtaaatactgccacccagatgccagttgcccaaccagctcctcctgtaacaacggtaaatgcctctgttctcatttccttggtggaagatgcgtaagccactcagaatgcaatgattcagtctgttgaggattggagtgaccagttccagcacataaagcaccatggtcccctggacaatcgtgttgttgaattgaatctgcccgtggcaggagtctgtcctacaaaataaaaatcataagccaaaaaaaaaaaaaaaaaaaaaaaaaaaaaaaaaaa

SEQ ID NO: 53 - protein, 99 aa, Conus ventricosus
MKIYLCLAVLLLLASTIVDSALHHKTEAIRNWRRKGRDENQCPECRCSELTTARCEESKYCHPDASCPTSSSCNNGKCLCSHFLGGRCVSHSECNDSVC

SEQ ID NO: 54 - DNA, 309 bp, Conus arenatus
atgaaaatttacctttgtcttgccattcttctgctcctggcttctaccatagtttattcagcgcttctcgataaagctgagactataagaaattggagacgcaaaatcagagatgaagcccaatgtgaagaatgcagatgcttagaattagtaatcccggaatgcactgaaagaaaatactgccacccagagcacagttgcccagtcagaccctactgtaacaacggtaaatgtctgtgtaaacatttctttggtggaagatgcgcaaagcgcccagactgcaatgatagtcactgtcgagcagagtag

SEQ ID NO: 55 - protein, 102 aa, Conus arenatus
MKIYLCLAILLLLASTIVYSALLDKAETIRNWRRKIRDEAQCEECRCLELVIPECTERKYCHPEHSCPVRPYCNNGKCLCKHFFGGRCAKRPDCNDSHCRAE

SEQ ID NO: 56 - DNA, 349 bp, Conus ermineus
caaaggaagagatgcaggccagtgtactggtggtacctgcagatgctccgaactaaaagaagccatgtgcgctgaaagtagattatgcctaagccccagttgcccatcaagctccgaatgtgcggacaatcattgcctctgtactcatatccttcatggagtatgcgtagactcgacagaatgcaaccctaacaagtgttgaggattggagtggccagttccagcacataaagcatcatggtctccttgacaatcgtgttgctgaaatgaatctacccatggcaggaatctgtcctacaaaataaaaatcataagtaaagaaaaaaaaaaaaaaaaaaaaaaaaaaaaa

SEQ ID NO: 57 - protein, 66 aa, Conus ermineus
KGRDAGQCTGGTCRCSELKEAMCAESRLCLSPSCPSSSECADNHCLCTHILHGVCVDSTECNPNKC

SEQ ID NO: 58 - DNA, 303 bp, Conus australis
atgggcagtatgaaaaagtacctgtgtcttgctattcttctgcttctggcttctaccataattgattcagggccttttgataaaattgagactataagaaagtggagacgcgatgaaagccagtgcggtgactgcagatgcttcgagttacgcaccgccagatgcacagataaacttcagtgcgagtcacccgacttgtgcaaacccgtcgtctcatgtgagactggtaaatgccactgtactcgtttcagttcgggatgttgcactagaaccgtggaatgcatgcctaaaaagtgtttctaa

SEQ ID NO: 59 - protein, 100 aa, Conus australis
MGSMKKYLCLAILLLLASTIIDSGPFDKIETIRKWRRDESQCGDCRCFELRTARCTDKLQCESPDLCKPVVSCETGKCHCTRFSSGCCTRTVECMPKKCF